(19) United States
(12) Patent Application Publication
(10) Pub. No.: US 2026/0242324 A1
(43) Pub. Date: Aug. 20, 2026

(54) FATTY ACID MIMETICS AS MODULATORS OF GPR40 AND/OR GPR120

(71) Applicant: Trustees of Dartmouth College, Hanover, NH (US)

(72) Inventor: Glenn C. Micalizio, Norwich, VT (US)

(21) Appl. No.: 19/472,008

(22) PCT Filed: Mar. 28, 2024
(86) PCT No.: PCT/US2024/022012

§ 371 Date: Oct. 2, 2025

Related U.S. Application Data
(60) Provisional application No. 63/493,892, filed on Apr. 3, 2023.
(60) Provisional application No. 63/517,565, filed on Aug. 3, 2023.
(60) Provisional application No. 63/615,052, filed on Dec. 27, 2023.

(51) Int. Cl. C07C 57/03; A61K 31/201; A61P 3/04; C07C 51/16; G01N 33/68

(52) U.S. Cl. CPC C07C 57/03; A61K 31/201; A61P 3/04; C07C 51/16; G01N 33/68

(57) ABSTRACT

The present disclosure relates to fatty acid mimetics, synthetic methods for preparing such compounds, and methods of using such compounds.

### CROSS REFERENCE TO RELATED APPLICATIONS

This patent application claims priority to U.S. Provisional Patent Application No. 63/493,892, filed on Apr. 3, 2023, to U.S. Provisional Patent Application No. 63/517,565, filed on Aug. 3, 2023, and to U.S. Provisional Patent Application No. 63/615,052, filed on Dec. 27, 2023, the entire contents of which are fully incorporated herein by reference.

### FEDERALLY SPONSORED RESEARCH OR DEVELOPMENT

This invention was made with government support under R01 GM134725 awarded by the National Institutes of Health. The government has certain rights in the invention.

### FIELD OF THE INVENTION

The present disclosure provides fatty acid mimetics and, in particular, oleic acid analogs having activity (e.g., agonistic activity) at GPR40 and/or GPR120.

### BACKGROUND OF THE INVENTION

Fatty acid receptors are drug targets for indications that include metabolic disease and obesity among others. For example, Free Fatty Acid receptors 1 (FFA1) and 4 (FFA4), also known as GPR40 and GPR120, respectively, are G-protein-coupled receptors (GPCR) responsive to medium- and long-chain fatty acids that are therapeutic targets for the treatment of metabolic diseases, including type 2 diabetes mellitus (T2DM).

Oleic acid is a conformationally heterogeneous fatty acid that has a regulatory effect at many different receptors, including GPR40 and GPR120. Agonism of GPR40 by oleic acid results in regulation of insulin secretion from pancreatic beta cells. However, the structural basis for this regulatory function of oleic acid at GPR40 remains undefined.

The structure of oleic acid is shown in FIG. 1.

Notably, the double bond present in oleic acid is the only moiety present that offers a conformational constraint for this molecule. Specifically, the stereochemistry of this functional group (the alkene) controls where the relative position of the two sp3 carbons bound to it will reside in 3-D space in relation to the plane of the double bond. Moving beyond those two sp3 carbons that are attached to the alkene, the vast majority of the hydrocarbon chains stemming from the central alkene are conformationally unorganized. As such, in the absence of structurally data that reveal how oleic acid binds to the receptor of interest (e.g. GPR40), it is not possible to accurately predict the conformation that oleic acid takes when bound.

Pharmaceutical efforts to drug GPR40 have resulted in scores of synthetic agents that are structurally unrelated to oleic acid. Many of these molecules, while quite potent, agonize GPR40 in ways that are “uninformed” with respect to the action of the natural agonist (i.e., they were not designed to mimic the manner in which oleic acid activates this receptor). Many pharmaceutical ligands are allosteric modulators of GPR40 that result in potent agonism of the receptor but do so in a manner that generates undesired toxicity. As a result, there are no FDA-approved agonists of GPR40 on the market today.

The molecular challenge addressed herein relates to the hopelessly unorganized conformation of natural fatty acids, and the typical phenomenological relationship between a particular fatty acid and a medically relevant receptor—a relationship that is informative with regard to function (e.g., agonist), but typically uninformed with respect to the molecular details of how the fatty acid ligand engages the targeted receptor (e.g., clearly defined binding mode that results in the observed function). Without structural data associated with the ligand-receptor complex (e.g., X-ray data from a fatty acid ligand-receptor complex), chemists looking to advance natural product-inspired therapeutics do not know which conformation of the hopelessly unorganized fatty acid is relevant for the desired function. As a result, drug discovery efforts often take the form of random screening efforts of compounds whose structures are not inspired by the endogenous ligan-receptor complex.

### SUMMARY OF THE INVENTION

The present disclosure relates to novel compounds and, in particular, fatty acid mimetics. In certain embodiments, the fatty acid mimetics are chiral and conformationally constrained enantioenriched variants of natural fatty acids. In certain embodiments, the fatty acid mimetic is a modulator (e.g., an agonist) of GPR40 and/or GPR120.

The present disclosure relates to fatty acid (e.g., oleic acid) mimetics having at least one and, preferably, at least two chiral conformational constraints. In certain embodiments, the fatty acid mimetic comprises a polar head, a first chiral conformational constraint (labeled as “region 1” in FIG. 2), a second chiral conformational constraint (“region 2”), a flexible subunit interspersed between the two conformational constraints, and a hydrophobic tail. A schematic structure of exemplary oleic acid mimetics is shown in FIG. 2.

### BRIEF DESCRIPTION OF THE DRAWINGS

FIG. 1 depicts the structure of oleic acid.

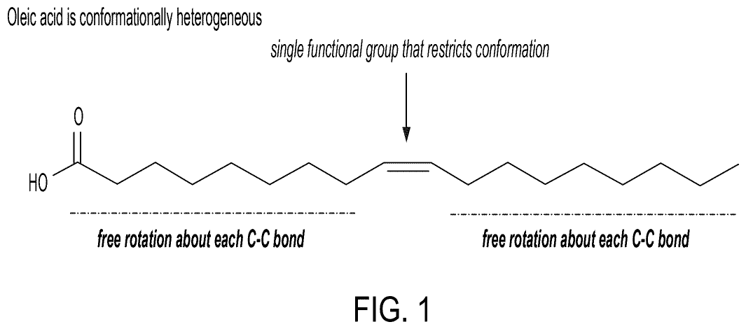

FIG. 2 depicts a schematic structure of exemplary oleic acid mimetics.

FIG. 3 depicts a collection of motifs that offer local conformational bias.

FIG. 4 depicts selected motifs to achieve rigidification.

FIG. 5 depicts four “classes” of mimetics that differ by the position of the conformational constraint.

FIG. 6A depicts chemistry capable of systematically making a fatty acid mimetic.

FIG. 6B depicts a retrosynthetic strategy for members of the fatty acid mimetic panel.

FIG. 7 depicts traces of a carbon backbone through a diamond lattice to provide examples of different conformations.

FIG. 8A depicts the structure of the alkynes and allylic alcohols employed.

FIG. 8B depicts examples of the chemical pathway for the synthesis of members of the fatty acid mimetic collection. Yield includes all isomers after deprotection: 21−rs=3:1, Z:E (of major regioisomer)=13:1; 22−rs=4:1, Z:E (of major regioisomer)=10:1; 23−rs=4:1, Z:E (of major regioisomer)=7:1, only one alkene stereoisomer visible via ¹HNMR for the minor regioisomeric coupling product.

FIG. 9A is a heat map showing % maximal efficacy of the collection for GPR40 and GPR120 (single dose at 15 μM).

FIG. 9B shows comparison of EC₅₀ values for heat-map-directed select compounds at GPR40 and GPR120.

FIG. 10A depicts exemplary 1,4-dienes and stability of intermediate radicals.

FIG. 10B depicts the metabolic stability in hepatocytes of selected mimetics versus positive control.

FIG. 11A depicts docking of 2-C, 4-C, and oleic acid to the GPR120 cryo-EM structure (PDB ID 8id6) stripped of the experimentally observed oleic acid molecule. Oleic acid and 2-C, with agonist activity against GPR120, show minimal residual mean square deviation (RMSD) of their aliphatic chains relative to the cryo-EM structure, while 4-C, which is inactive against GPR120, has a higher RMSD.

FIG. 11B depicts conformational analysis of oleic acid and 2-C from molecular dynamics simulations of the unbound molecules in solution. The radar plots illustrate the dihedral angles populated at each bond depicted through molecular dynamics simulations. Several bond dihedrals in 2-C show conformational bias toward the angles experimentally observed in the GPR120-bound oleic acid conformation. Bond dihedrals not shown in the figure show similar distributions between oleic acid and compound 2-C.

### DESCRIPTION OF THE INVENTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

In one aspect, the present disclosure relates to a fatty acid mimetic comprising a conformationally constrained core comprising two or more conformational constraints and at least one optional flexible subunit. In some such embodiments, the flexible subunit (e.g., an alkylene, such as a methylene) is interspersed between the two conformational constraints.

In certain embodiments, the hydrocarbon length of oleic acid (18) is a common characteristic of the mimetic.

In one aspect, the present disclosure relates to a compound of Formula (I) or a pharmaceutically acceptable salt thereof, wherein A, X, R, m and n are as defined below.

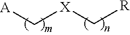

In one aspect, the present disclosure relates to a compound of Formula (II) or a pharmaceutically acceptable salt thereof, wherein X, R¹, m and n are as defined below.

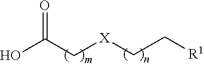

In one aspect, the present disclosure relates to a compound of Formula (III) or a pharmaceutically acceptable salt thereof, wherein X¹, X², m and n are as defined below.

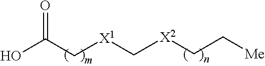

### A. Definitions

As used in the specification and the appended claims, unless specified to the contrary, the following terms have the meaning indicated:

As used herein, the terms “a,” “an,” and “the” include both singular and plural referents, unless the context clearly dictates otherwise. Thus, for example, “a compound” includes one compound and plural compounds. The terms “first” and “second” are terms to distinguish different elements, not terms supplying a numerical limit, and a device having first and second element can also include a third, a fourth, a fifth, and so on, unless otherwise indicated.

As disclosed herein, numeric ranges are provided for various parameters or data. It should be understood that numeric ranges also disclose include each intervening value within the range, to the tenth of the unit of the lower limit, unless the context clearly dictates otherwise. It should also be understood that the upper and lower limits may each be included or excluded in a range.

It should be recognized that chemical structures and formula may be elongated or enlarged for illustrative purposes.

The term “about” as used herein means approximately, and in most cases within 10% of the stated value.

The term “substituted” in reference to alkyl, heteroalkyl, alkenyl, heteroalkenyl, alkynyl, heteroalkynyl, aryl, heteroaryl, heterocycle etc., for example, “substituted alkyl”, “substituted heteroalkyl”, “substituted alkenyl”, substituted heteroalkenyl”, “substituted alkynyl”, “substituted heteroalkynyl”, “substituted aryl”, “substituted heteroaryl”, and “substituted heterocycle” means alkyl, heteroalkyl, alkenyl, heteroalkenyl, alkynyl, heteroalkynyl, aryl, heteroaryl, heterocycle, respectively, in which one or more hydrogen atoms are each independently replaced with a non-hydrogen substituent or with a deuterium atom. Typical substituents include, but are not limited to, —R¹², —R¹⁴, —O—, ═O, —OR¹⁴, —SR¹⁴, —S—, —NR¹⁴₂, —N⁺R¹⁴₃, ═NR¹⁴, —C(R¹²)₃, —CN, —OCN, —SCN, —N═C═O, —NCS, —NO, —NO₂, ═N₂, —N₃, —NHC(═O)R¹⁴, —NHS(═O)₂R¹⁴, —C(═O)R¹⁴, —C(═O)N(R¹⁴)₂—S(═O)₂O⁻, —S(═O)₂₀H, —S(═O)₂R¹⁴, —OS(═O)₂OR¹⁴, —S(═O)₂NR¹⁴, —S(═O)R¹⁴, —OP(═O)(OR¹⁴)₂, —P(═O)(OR¹⁴)₂, —P(═O)(O—)₂, —P(═O)(OH)₂, —P(O)(OR¹⁴)(O—), —C(═O)R¹⁴, —C(═O)OR¹⁴, —C(═O)R¹², —C(S)R¹⁴, —C(O)OR¹⁴, —C(O)O—, —C(S)OR¹⁴, —C(O)SR¹⁴, —C(S)SR¹⁴, —C(O)N(R¹⁴)₂, —C(S)N(R¹⁴)₂, —C(═NR¹⁴)N(R¹⁴)₂, where each R¹² is independently a halogen: F, Cl, Br, or I; and each R¹⁴ is independently hydrogen, deuterium, alkyl, aryl, arylalkyl, a heterocycle, or a protecting group. When the number of carbon atoms is designated for a substituted group, the number of carbon atoms refers to the group, not the substituent (unless otherwise indicated). For example, a C₁₋₄ substituted alkyl refers to a C₁₋₄ alkyl, which can be substituted with groups having more than, e.g., 4 carbon atoms.

The terms “alkyl” and “alkylene” refer to a straight-chain or branched alkyl, preferably having 1, 2, 3, 4, 5, 6, 7, 8, 9, 10, 11, 12, 13, 14, 15, 16, 17, 18, 19, 20, 21, 22, 23 or 24 carbons. Examples of such alkyl groups include methyl, ethyl, propyl, isopropyl, n-butyl, sec-butyl, isobutyl, tert-butyl, pentyl, isoamyl, hexyl, and the like. Alkyl groups may be unsubstituted or substituted, as defined above.

The term “alkenyl” refers to a straight or branched hydrocarbon, preferably having 2, 3, 4, 5, 6, 7, 8, 9, 10, 11, 12, 13, 14, 15, 16, 17, 18, 19, 20, 21, 22, 23 or 24 carbons, and having one or more carbon-carbon double bonds. Nonlimiting examples of alkenyl groups include ethenyl, 1-propenyl, 2-propenyl (allyl), iso-propenyl, 2-methyl-1-propenyl, 1-butenyl, and 2-butenyl. Alkenyl groups may be unsubstituted or substituted by one or more suitable substituents, as defined above.

The term “alkynyl” refers to a straight or branched hydrocarbon, preferably having 2, 3, 4, 5, 6, 7, 8, 9, 10, 11, 12, 13, 14, 15, 16, 17, 18, 19, 20, 21, 22, 23 or 24 carbons, and having one or more carbon-carbon triple bonds. Alkynyl groups include, but are not limited to, ethynyl, propynyl, and butynyl. Alkynyl groups may be unsubstituted or substituted by one or more suitable substituents, as defined above.

Whenever a range of the number of atoms in a structure is indicated (e.g., a C₁-C₂₄ alkyl, C₂-C₂₄ alkenyl, C₂-C₂₄ alkynyl, etc.), it is specifically contemplated that the structure can have any individual number of carbon atoms falling within the indicated range. By way of example, a description of the group such as an alkyl group using the recitation of a range of 1-25 carbon atoms (e.g., C₁-C₂₅) encompasses and specifically describes an alkyl group having any of 1, 2, 3, 4, 5, 6, 7, 8, 9, 10, 11, 12, 13, 14, 15, 16, 17, 18, 19, 20, 21, 22, 23, 24 and 25 carbon atoms. It is also contemplated that any sub-range can be formed from such a range. As examples, a reference to a C₁-C₂₅ alkyl, or a reference to m or n being 1 to 25 in Formulas (I, II, and III), should be understood as disclosing the sub-ranges of 1-7 carbon atoms (e.g., C₁-C₇), 1-6 carbon atoms (e.g., C₁-C₆), 1-4 carbon atoms (e.g., C₁-C₄), 1-3 carbon atoms (e.g., C₁-C₃), or 2-24 carbon atoms (e.g., C₂-C₂₄) as well.

“Heteroalkyl”, “heteroalkenyl” and “heteroalkynyl” refers respectively to a molecule comprising an alkyl group, an alkenyl group and an alkynyl group, in which one or more carbon atoms have been replaced with a heteroatom, such as, O, N, or S, except that the carbon atom of the alkene or alkyne are not replaced. Other carbons within the molecule, including carbons of an alkyl group, can be replaced independently with a heteroatom (O, N, or S), meaning the first carbon, the terminal carbon or an internal carbon. A “substituted heteroalkyl”, a “substituted heteroalkenyl”, or a “substituted heteroalkynyl” refers to a molecule comprising a heteroalkyl, a heteroalkenyl or a heteroalkynyl group as defined herein, and one or more hydrogen atom has been replaced with a substituent (as defined above in the “substituted” definition).

The term “aryl” refers to an unsubstituted or substituted aromatic carbocyclic substituent, such as phenyl, naphthyl, anthracyl, indanyl, and the like. It is understood that the term aryl applies to cyclic substituents that are planar and comprise 4n+2 electrons, according to Hückel's Rule. Aryl groups may be unsubstituted or substituted by one or more suitable substituents, as defined above, and include variants that are polycyclic wherein at least one of rings is aromatic. The term “heteroaryl” refers to a monocyclic or bicyclic 5- or 6-membered ring system, wherein the heteroaryl group is unsaturated and satisfies Hückel's rule.

The term “carbonyl” refers to a substituent comprising a carbon double bonded to an oxygen. Examples of such substituents include aldehydes, ketones, carboxylic acids, esters, amides, carbonates, and carbamates. Carbonyl groups may be unsubstituted or substituted by one or more suitable substituents, as defined above.

The term “amino” refers to any nitrogen-containing moiety. Non-limiting examples of the amino group are NH₂— (primary), R¹⁵HN— (secondary), and (R¹⁵)₂N— (tertiary) where R¹⁵ is alkyl, alkenyl, alkynyl, aryl, heterocyclic, or heteroaryl.

The term “deuterated” refers to one or more hydrogen atoms being replaced by deuterium. For example, a deuterated alkyl is an alkyl wherein one or more hydrogen atoms in the alkyl group are replaced by deuterium. A deuterated group can be fully deuterated, wherein all hydrogens in the group are replaced by deuterium. Alternatively a deuterated group can be partially deuterated, wherein fewer than all hydrogens are replaced by deuterium. In the present disclosure, deuterium may be denoted by d or D, and a methyl group where all the three hydrogen atoms are replaced by deuterium atoms may be denoted by methyl-d3, d3-methyl, fully deuterated methyl, or CD₃.

The term “pharmaceutically acceptable” is used adjectivally to mean that the modified noun is appropriate for use as a pharmaceutical product for human use or as a part of a pharmaceutical product for human use.

“Halogen” or “halo” refers to fluorine, chlorine, bromine, and iodine.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

All patents and publications referred to herein are expressly incorporated by reference.

### B. Compounds

In one aspect, the disclosure provides a compound of Formula (I):

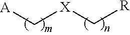

or a pharmaceutically acceptable salt thereof; wherein A comprises a polar head group; X comprises a conformationally constrained core comprising two or more conformational constraints and at least one optional flexible subunit; R comprises a non-polar group, generally intended to mimic the non-polar tail of an endogenous fatty acid ligand; and each of m and n is independently an integer from 1 to 25, or from 1 to 19, or from 1 to 11, or from 1 to 7.

In certain embodiments, the flexible subunit is an alkylene. In some such embodiments, the flexible subunit is a methylene subunit.

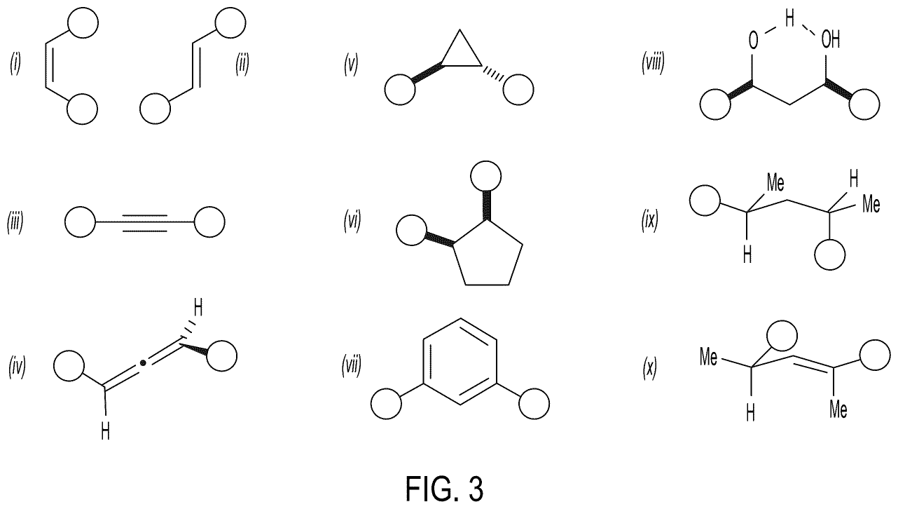

The conformational constraints are inspired by the structural motifs shown in FIG. 3. The conformational constraints depicted in FIG. 3 can be characterized as acyclic conformational constraints (e.g., (i), (ii), (iii), (iv), (ix), and/or (x)); simple π-unsaturation conformational constraints (e.g., (i)-(iv)); acyclic chiral conformational constraints (e.g., (ix) and/or (x)); and cyclic conformational constraints (e.g., (v)-(viii) where it is understood that saturated and unsaturated variants would be useful). The circles indicate the conformational constraint's points of attachment to other portions of the compound.

In some embodiments, the compound has a structure of Formula (I-A):

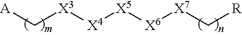

wherein
X³ is a bond, an alkyl, or a conformational constraint;
X⁴ is a bond or a flexible subunit;
X⁵ is a conformational constraint;
X⁶ is a bond or a flexible subunit; and
X⁷ is a bond, an alkyl, or a conformational constraint.
   In some embodiments, at least one of X³ or X⁷ is a conformational constraint, such as one of the conformational constraints (i) to (x) in FIG. 3 above. In some embodiments, at least one of X³ or X⁷ (or both of X³ or X⁷) is an acyclic conformational constraint (e.g., (i), (ii), (iii), (iv), (ix), and/or (x) about); or a simple π-unsaturated conformational constraint (e.g., (i)-(iv)); or an acyclic chiral conformational constraints (e.g., (ix) and/or (x)); or a cyclic conformational constraint (e.g., (v)-(viii)). In some embodiments, at least one of X³ or X⁷ (or both of X³ and X⁷) is a conformational constraint comprising a ring system. In some embodiments, at least one of X³ or X⁷ (or both of X³ and X⁷) is a cycloalkyl, such as (For example) 1,2-cis-disubstituted cyclopentane, 1,2-trans-disubstituted cyclopentane, cis-substituted cyclopropanes, trans-substituted cyclopropanes, or 1,3-cis-disubstituted tetrahydrofuran. In some embodiments, X⁴ and X⁶ are independently C₁₋₆-alkylene, such as a C₁₋₃-alkylene (e.g., methylene, ethylene, or propylene).

A comprises a polar head group. In some embodiments, A consists of a polar head group. In some embodiments, A comprises A²-R¹⁵, wherein A² is a polar head group, and R¹⁵ is a bond, a C₁₋₆-alkyl, or a C₂₋₆-alkenyl. In some embodiments, A or A² comprises a carboxylic acid, a carboxyl ester, an amide, a ketone, a phosphonic acid, a phosphinic acid, a sulfonic acid, a sulfinic acid, a sulfonamide, an acyl-sulfonamide, a hydroxamic acid, a hydroxamic ester, a sulfonylurea, an acylurea, a tetrazole, a thiazolidinedione, an oxazolidinedione, an oxadiazolone, a thiadiazolone, an oxathiadiazole oxide, an oxadiazolethione, an isoxazole, a tetramic acid, a cyclopentane diones, a phenol derivative, a squaric acid derivative, or a salt thereof. Exemplary polar head groups include, but are not limited to, those in Table 1 (wherein the circle indicates the groups point of attachment to the rest of the compound of Formula (I or I-A):

TABLE 1

| | |
|---|---|
| Carboxylic acids | 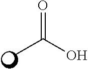 |
| Carboxyl esters | 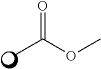 |
| Amides | 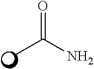 |
| Ketones | 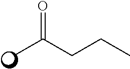 |
| Hydroxamic acids | 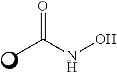 |
| | 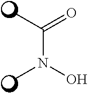 |
| Hydroxamic esters | 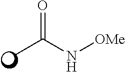 |
| | 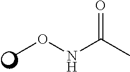 |
| Phosphonic acid | 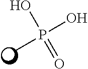 |
| Phosphinic acid | 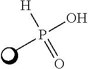 |
| Sulfonic acid (sodium salt) | 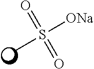 |
| Sulfinic acid (sodium salt) | 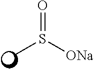 |
| Sulfonamides | 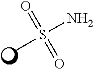 |
| | 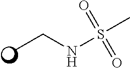 |
| Acyl-sulfonamides | 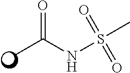 |
| | 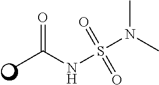 |
| Sulfonylurea | 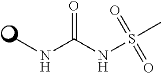 |
| Acylurea | 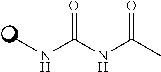 |
| Tetrazole | 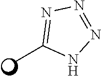 |
| Thiazolidine dione | 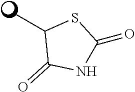 |
| Oxazolidine dione | 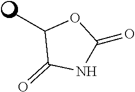 |
| Oxadiazol-5(4H)-one | 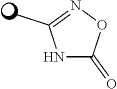 |
| Thiadiazol-5(4H)-one | 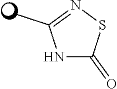 |
| Oxathiadiazole-2-oxide | 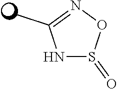 |
| Oxadiazol-5(4H)-thione | 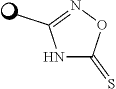 |
| Isoxazole | 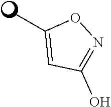 |
| Tetramic acid | 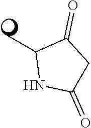 |
| Cyclopentane 1,3-dione | 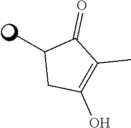 |
| | 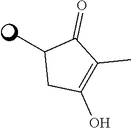 |
| | 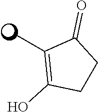 |
| Cyclopentane 1,2-diones | 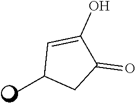 |
| Squaric acid derivatives | 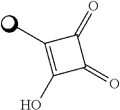 |
| | 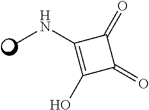 |
| Substituted phenols | 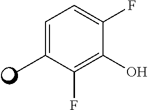 |
| | 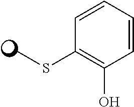 |
| | 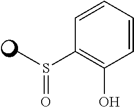 |
| | 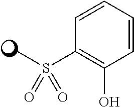 |

In one aspect, the disclosure provides a compound of Formula (II):

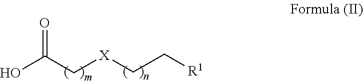

or a pharmaceutically acceptable salt thereof; wherein:
   X comprises a constrained core comprising one or more acyclic conformational constraints and at least one optional flexible subunit;
   R¹ is a C₁₋₆-alkyl; and
   each of m and n is independently an integer from 1 to 25, or from 1 to 19, or from 1 to 11, or from 1 to 7;
   provided that the compound is not a natural product and/or
   provided that at least one conformational constraint is a conformational constraint other than

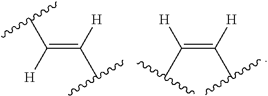

In certain embodiments, the constrained core comprises (ix):

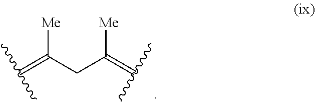 [SMILES: *=C(C)CC(=*)C]

In certain embodiments, the acyclic conformational constraints are independently are selected from the group consisting of (x), (x′), (x″), and (x′″):

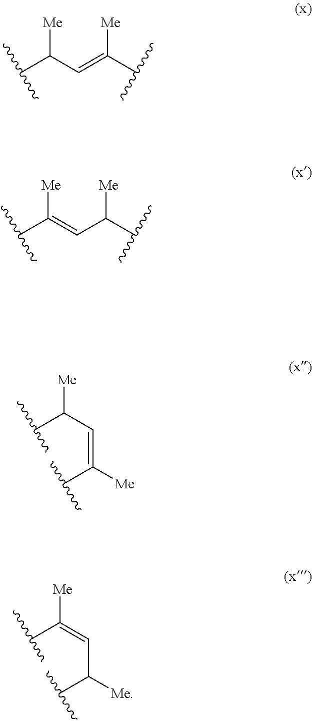 [SMILES: */C(C)=C/C(*)C ; */C(C)=C/C(*)C ; */C(C)=C\C(*)C ; */C(C)=C\C(*)C]

In certain embodiments, two or more conformational constraints are joined by a C₁₋₆-alkylene, such as a C₁₋₃-alkylene (e.g., methylene, ethylene, or propylene).

In one aspect, the disclosure provides a compound of Formula (III):

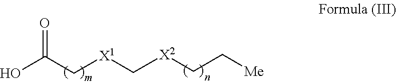

or a pharmaceutically acceptable salt thereof; wherein:
X¹ is

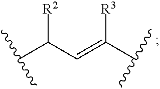

R² is C₁₋₆-alkyl;
R³ is C₁₋₆-alkyl;
X² is

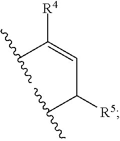

R⁴ is C₁₋₆-alkyl;
R⁵ is hydrogen or C₁₋₆-alkyl; and
each of m and n is independently an integer from 1 to 25, or from 1 to 19,
or from 1 to 11, or from 1 to 7.

In Formulas (I), (II) and (III), each of m and n can independently be 1, 2, 3, 4, 5, 6, 7, 8, 9, 10, 11, 12, 13, 14, 15, 16, 17, 18, 19, 20, 21, 22, 23, 24, or 25. In certain embodiments, m is 1, 3, 5, 7, 9, 11, 13, 15, 17, 19, 21, 23 or 25. In certain embodiments of the Formulas, n is 1, 3, 5, 7, 9, 11, 13, 15, 17, 19, 21, 23 or 25. In certain embodiments, m+n is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40.

In certain embodiments, X¹ is (x.1) or (x.2):

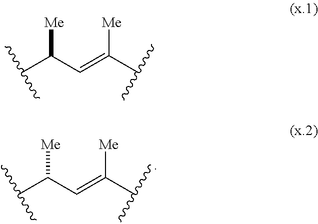 [SMILES: */C(C)=C/[C@@H](*)C ; */C(C)=C/[C@H](*)C]

In certain embodiments, X² is (x′″.1) or (x′″.2):

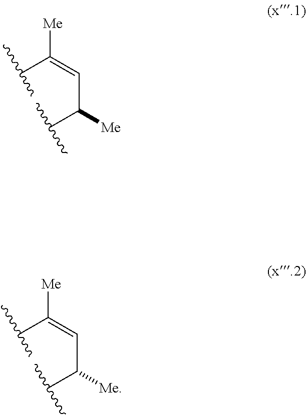 [SMILES: */C(C)=C\[C@H](*)C ; */C(C)=C\[C@@H](*)C]

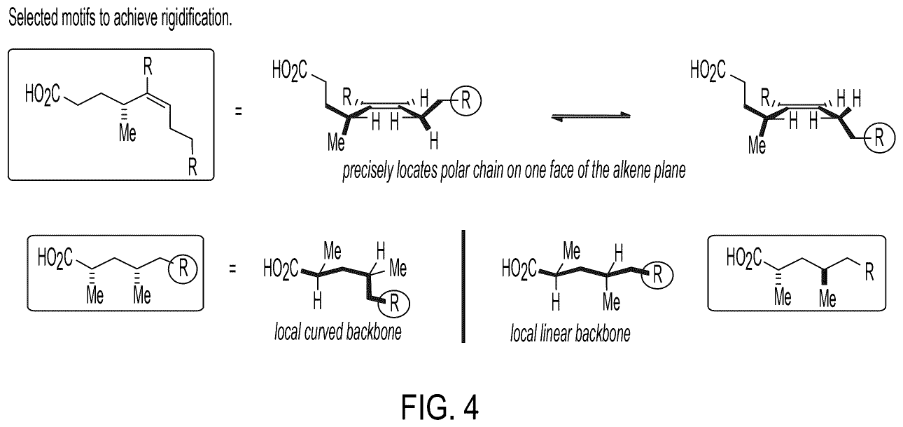

In certain embodiments for any aspect of the invention, the nature of the chiral conformational constraint is based on a structural motif observed in polyketide natural product such as those shown in FIG. 4 (avoiding allylic strain and eclipsing 1,5-interactions (aka, syn-pentane interactions)).

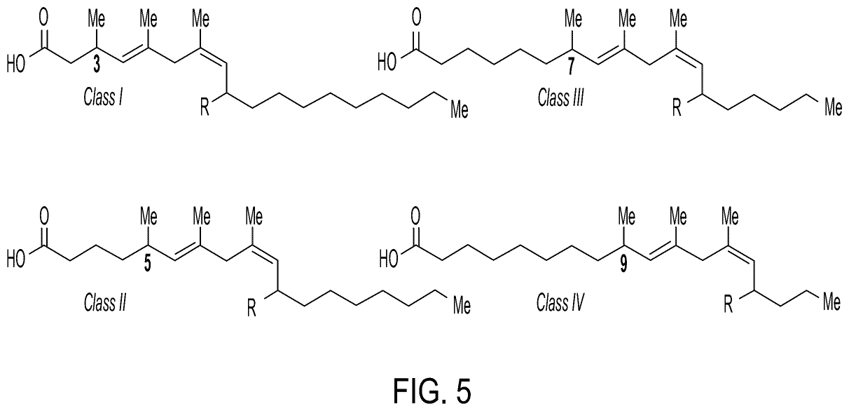

Exemplary compounds include Class I, II, III, IV compounds shown in FIG. 5 where “R” is a hydrogen or C₁₋₆-alkyl. Notably, the conformational constraints within each of these “classes” are positioned at different locations within the fatty acid carbon backbone (for “Class I”, the constraint begins at C3, for “Class II”, the constraint begins at C5, for “Class III”, the constraint begins at C7, and for “Class IV”, the constraint begins at C9).

In one aspect, compounds disclosed herein possess biological activity, for example, by modulating a fatty acid receptor, such as GPR40 and/or GPR120.

### C. Methods of Synthesis

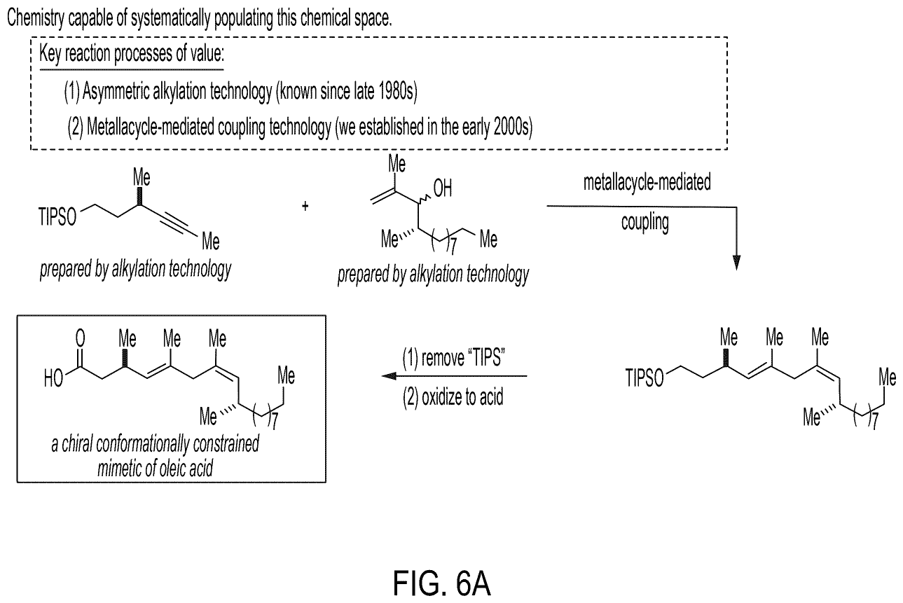

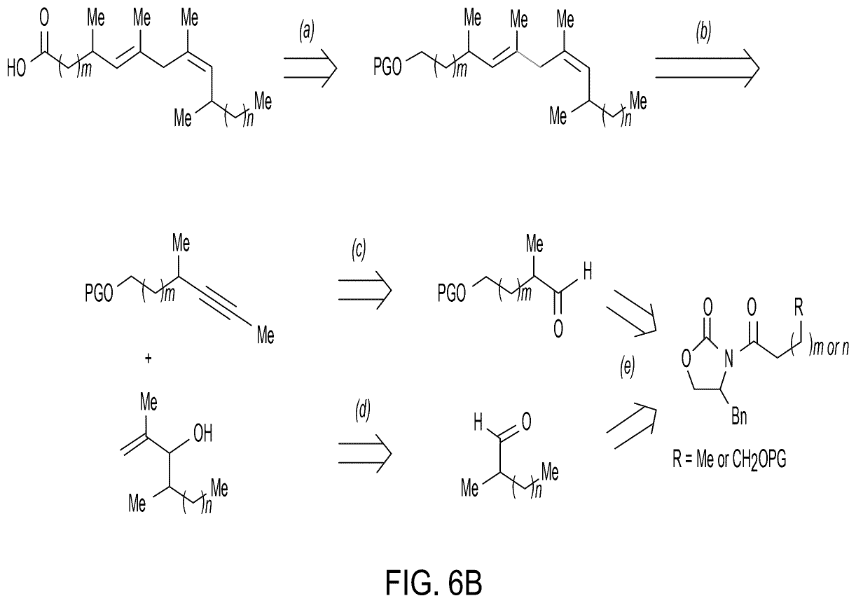

As summarized in FIG. 6A the chemistry to make a fatty acid mimetic leveraged asymmetric alkylation chemistry well known to those skilled in the art, and included a modern metallacycle-mediated coupling reaction between an alkyne and an allylic alcohol. The synthetic pathway is modular/convergent and allowed for synthesis of fatty acid mimetics that contain the type of conformational constraints depicted in FIG. 6A at different positions within a fatty acid mimetic (in the case depicted, the compounds were designed to contain a C₁₈ backbone, inspired by oleic acid).

In one aspect, this disclosure provides a method for making a compound of any of Formulas (I), (II) or (III), such as the fatty acid mimetics described herein. The method comprises coupling of an alkyne and an allylic alcohol, such as by metallacycle-mediated coupling. One skilled in the art may contemplate alternative methods for the construction of such targets in light of the teachings of the present disclosure, including the use of Pd-catalyzed cross-coupling technology (albeit with the understanding the nature of the coupling partners would have to be compatible with such transition metal catalyzed coupling chemistry).

In some embodiments, a method is provided for making a compound of Formula (I), wherein the method comprises coupling an alkyne of Formula (IV):

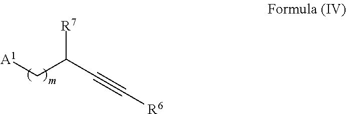

wherein A¹ comprises a precursor to a polar head group (like a protected alcohol) or the actual targeted polar head group; R⁶ is a C₁₋₆-alkyl; R⁷ is hydrogen or a C₁₋₆-alkyl; with an allylic alcohol of Formula (V):

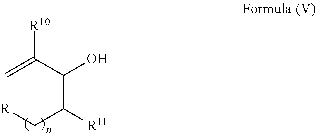

wherein R comprises a group intended to mimic the non-polar tail of endogenous fatty acids; R¹⁰ is C₁₋₆-alkyl; R¹¹ is hydrogen or a C₁₋₆-alkyl; and each of m and n is independently an integer from 1 to 25, or from 1 to 19, or from 1 to 11, or from 1 to 7. The coupling can be a metallacycle-mediated coupling, which can be performed in the presence of a titanium alkoxide, such as Ti(Oi-Pr)₄, and an organometallic reductant [such as n-butyl lithium (n-BuLi) or i-PrMgX)], and can be conducted in the presence of a silylating agent, such as trimethylsilyl chloride (TMSCl).

Additional information regarding metallacycle-mediated coupling and synthesis of 1,4-dienes in a convergent fashion can be found in (a) Kolundzic, F.; Micalizio, G. C.; Synthesis of Substituted 1,4-Dienes by Direct Alkylation of Allylic Alcohols. J. Am. Chem. Soc. 2007, 129, 15112. (b) Diez, P. S.; Micalizio G. C. Chemoselective Reductive Cross-Coupling of 1,5-Diene-3-ols with Alkynes: A Facile Entry to Stereodefined Skipped Trienes. J. Am. Chem. Soc. 2010, 132, 9576-9578. (c) Jeso, V.; Micalizio G. C. Total Synthesis of Lehualide B by Allylic Alcohol-Alkyne Reductive Cross-Coupling. J. Am. Chem. Soc. 2010, 132, 11422-11424. (d) Diez, P. S.; Micalizio, G. C. Convergent Synthesis of Deoxypropionates. Angew. Chem. Int. Ed. Engl. 2012, 51, 5152-5156.

Exemplary silylating agents include, but are not limited to trimethylsilyl chloride (TMSCl), trimethylsilyl bromide (TMSBr), and chlorotriethylsilane (TESCl).

In some embodiments, the alkyne has a structure of Formula (IV-A):

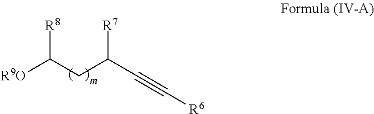

wherein R⁸ is hydrogen or a C₁₋₆-alkyl; and R⁹ is hydrogen or an oxygen protecting group. In some embodiments, the compound has a structure of Formula (IV-A-1 or (IV-A-2):

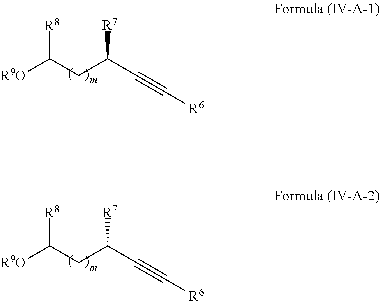

wherein R⁷ is a C₁₋₆-alkyl. In some embodiments of Formulas (IV-A, IV-A-1, or IV-A-2), R⁸ is hydrogen; and R⁹ is an oxygen protecting group. In some embodiments, R⁹ is an oxygen protecting group selected from methyl, tert-butyloxycarbonyl (BOC), methoxymethyl (MOM), tert-butyldimethylsilyl (TBS), tert-butyldiphenylsilyl (TBDPS), tribenzylsilyl, and triisopropylsilyl (TIPS).

Examples of oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), t-butoxymethyl, 4-pentenyloxymethyl (POM), 2-methoxyethoxymethyl (MEM), tetrahydropyranyl (THP), 2-trimethylsilylethyl, t-butyl, p-chlorophenyl, p-methoxyphenyl, benzyl (Bn), p-methoxybenzyl, 3,4-dimethoxybenzyl, p-halobenzyl, 2,6-dichlorobenzyl, p-phenylbenzyl, diphenylmethyl, 5-dibenzosuberyl, triphenylmethyl, trimethylsilyl (TMS), triethylsilyl (TES), triisopropylsilyl (TIPS), dimethylisopropylsilyl (IPDMS), diethylisopropylsilyl (DEIPS), t-butyldimethylsilyl (TBDMS), t-butyldiphenylsilyl (TBDPS), tribenzylsilyl, triphenylsilyl, diphenylmethylsilyl (DPMS), 2-methoxyethoxymethyl (MEM), 2-trimethylsilylethoxymethyl (SEM), methylthiomethyl (MTM), methoxytrityl (MMT), and dimethoxytrityl (DMT).

In some embodiments, the present methods further comprise making the alkyne by carbonyl alkynylation of a compound of Formula (VI or VI-A):

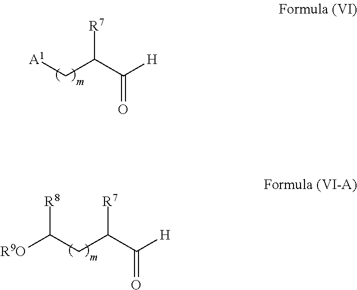

In some embodiments, the allylic alcohol has a structure of Formula (V-A or V-A-1 or V-A-2):

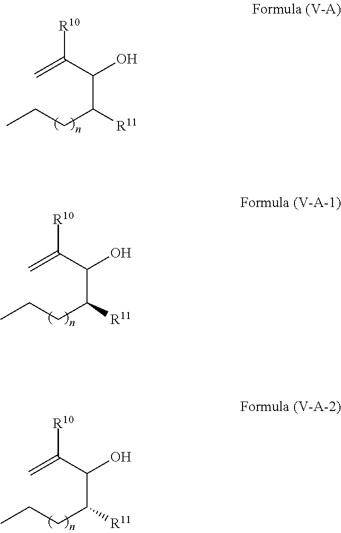

wherein R¹¹ is a C₁₋₆-alkyl. The stereochemistry at the allylic position is generally not critical and can be selected by those practicing the method. In some embodiments, the allylic position is a mixture of stereoisomers. In some embodiments, the allylic alcohol is enriched at the allylic position as mostly (S) or (R).

In some embodiments, the method further comprises making the allylic alcohol of Formula (V or V-A) by reaction of a compound of Formula (VII or VII-A) to convert aldehyde to allylic alcohol in a targeted manner:

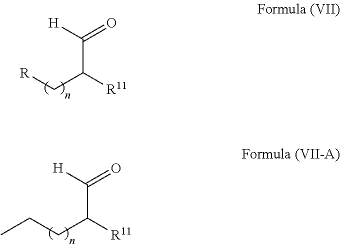

There are many available methods or the addition of vinyl groups to aldehydes, including other vinyl organometallic reagents used stoichiometrically or generated catalytically (e.g. NHK-coupling). Accordingly, the method can comprise making the allylic alcohol of Formula (V or V-A) by a reaction which comprises (a) a reaction with a Grignard reagent, e.g., (R¹⁰)(CH₂)C—MgR¹², where R¹² is a halogen; (b) a reaction with a vinyl lithium reagent; (c) catalytic addition by NHK coupling, or (d) reaction of an organolanthanum or organocerium reagent to an aldehyde.

In some embodiments, the present methods further comprise making the compound of any of Formulas (VI, VI-A, VII, or VII-A) by stereoselective alkylation of a compound of Formula (VIII) by methods known to those skilled in the art:

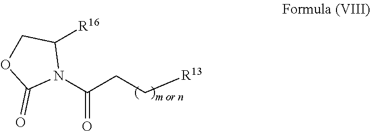

wherein R¹³ is C₁₋₆-alkyl or A¹; and R¹⁶ is a hydrogen, alkyl, aryl or arylalkyl.

In some embodiments, the present methods comprise stereoselectively alkylating a compound of Formula (VIII) wherein R¹³ is C₁₋₆-alkyl. After removal of the auxiliary and altering the oxidation state of the carbon previously attached to nitrogen by methods well known in the art, one can arrive at a compound of Formula (VII):

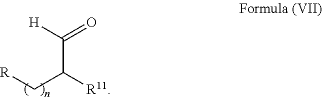

The compound of Formula (VII) is reacted with (R¹⁰)(CH₂)C—MgR¹² in a Grignard reaction to form the allylic alcohol of Formula (V). The methods also comprise stereoselectively alkylating a compound of Formula (VIII) wherein R¹³ is A¹, to form a compound of Formula (VI):

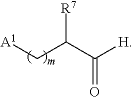

The methods also comprise making the alkyne of Formula (IV) by carbonyl alkynylation of the compound of Formula (VI). The present methods can then comprise coupling the alkyne of Formula (IV) and the allylic alcohol of Formula (V) as described above.

In some embodiments, A¹ comprises a precursor to the desired polar head group suitably protected with a protecting group (e.g., triisopropylsilyl), and the method further comprises deprotecting and oxidizing the head group to form a carboxylic acid group.

In one aspect, a method for making the compound is based on the retrosynthetic strategy depicted in FIG. 6B, where (a)=deprotection/oxidation, (b)=alkyne-allylic alcohol coupling, (c)=carbonyl alkynylation, (d)=Grignard addition, and (e)=Evans' diastereoselective alkylation.

### D. Methods of Use

In one aspect, this disclosure provides a method for treatment of a disease or condition mediated by the activation of GPR40 and/or GPR120 in a subject in need thereof. The method includes the step of administering to the subject, preferably a human, a fatty acid mimetic described herein.

Diseases and conditions treatable with agonists of GPR40 and/or GPR120 include, but are not limited to, diabetes mellitus, in particular Type 2 diabetes, Type 1 diabetes, complications of diabetes (such as, for example, retinopathy, nephropathy or neuropathies, diabetic foot, ulcers or macroangiopathies), metabolic acidosis or ketosis, reactive hypoglycaemia, hyperinsulinaemia, glucose metabolic disorder, insulin resistance, metabolic syndrome, dyslipidaemias of different origins, atherosclerosis and related diseases, obesity, high blood pressure, chronic heart failure, oedema and hyperuricaemia.

In one aspect, this disclosure provides a method for treatment of metabolic diseases, such as type 2 diabetes mellitus, as well as conditions associated with this disease, including insulin resistance, obesity, cardiovascular disease, and dyslipidemia.

Metabolic diseases may be classified as either congenital (caused by an inherited enzyme abnormality) or acquired (caused by a diseased endocrine organ or a failed metabolically important organ such as the liver or the pancreas). Diabetes mellitus is a metabolic disease, which is defined as a chronic hyperglycemia associated with consequential damages to organs and dysfunctions of metabolic processes. Diabetes mellitus Type 1 (a.k.a. insulin-dependent diabetes mellitus, IDDM) is a common disease in young people under 20 years of age.

In another aspect, this disclosure provides fatty acid mimetics, including GPR40 and/or GPR120 agonists, for use in a therapeutic method as described hereinbefore and hereinafter.

In one aspect, this disclosure provides fatty acid mimetics, including GPR40 and/or GPR120 agonists, in particular for the treatment of metabolic disorders, for example diabetes, dyslipidemia and/or obesity.

In certain embodiments, the fatty acid mimetics disclosed herein are provided in a pharmaceutical composition. Such pharmaceutical compositions comprises one or more fatty acid mimetics, optionally together with one or more inert carriers and/or diluents.

### E. Kits and Sets of Compounds

In another aspect, this disclosure provides kits or sets of two or more fatty acid mimetics. Each of the fatty acid mimetics in the kit or set is a compound of formula (I), (II), or (III) or a pharmaceutically acceptable salt thereof. In some embodiments, each of the fatty acid mimetics in the kit or set is a compound of formula (I) or a pharmaceutically acceptable salt thereof. each of the fatty acid mimetics in the kit or set is a compound of formula (II) or a pharmaceutically acceptable salt thereof. In some embodiments, each of the fatty acid mimetics in the kit or set is a compound of formula (III) or a pharmaceutically acceptable salt thereof.

In some embodiments of the kits or sets, each of the fatty and acid mimetics is in a separate vessel. The vessel(s) in which the fatty and acid mimetics are supplied can be any conventional container that is capable of holding the supplied form, for instance, microfuge tubes, microtiter plates, ampules, bottles, or integral testing devices, such as fluidic devices, cartridges, lateral flow, or other similar devices.

The kits or sets can also include packaging materials for holding the vessels or combination of vessels. Typical packaging materials for such kits and systems include solid matrices (e.g., glass, plastic, paper, foil, micro-particles and the like) that hold the fatty and acid mimetics in any of a variety of configurations (e.g., in a vial, microtiter plate well, microarray, and the like). The kits may further include instructions recorded in a tangible form for use of the fatty and acid mimetics.

In some embodiments, each of the fatty acid mimetics is conformationally constrained due to their precise molecular structures, and each of the mimetics in the set has a different structure from each of the other mimetics in the set. The kits or sets can have any number of fatty acid mimetics, such as at least 4, 6, 12 or 24 or more fatty acid mimetics. In some embodiments, each of the fatty acid mimetics is a mimetic of (a) a C12 fatty acid; (b) a C14 fatty acid; (c) a C16 fatty acid; (d) a C18 fatty acid; (e) a C20 fatty acid; or (f) a C22 fatty acid. As an example, a kit or set of C18 fatty acid mimetics can include each of Compounds 1-A to 4-F in separate vessels.

### F. Methods of Conformation Profiling

In another aspect, this disclosure provides methods of conformational profiling as a means to narrow the conformational space relevant for a particular function of a targeted fatty acid. The method can comprise making a set of mimetics of the target fatty acid; contacting the mimetics with the fatty acid receptor; and assessing function of each fatty acid as determined by the resulting activity of the receptor or other macromolecular target.

The information gleaned from this profiling identifies subpopulations within the collection of mimetics that have structural features that are preferred for achieving the desired function. Further structural rigidification of the identified leads is appreciated as a means to optimize ligands that surface from such conformational profiling.

The mimetics of the set will have a plurality of different structures. In some embodiments, the set has at least 6 different structures, or at least 12, 24, 36, 48 or 96 different structures. In some embodiments, each of the fatty acid mimetics is a mimetic of (a) a C12 fatty acid; (b) a C14 fatty acid; (c) a C16 fatty acid; (d) a C18 fatty acid; (e) a C20 fatty acid; or (f) a C22 fatty acid. For example, where each of the mimetics of the set is a C18 fatty acid mimetic, the set can comprise each of Compounds 1-A to 4-F.

In the present methods, function can be assessed by any suitable technique. In some embodiments, function is assessed in a biological assay, such a β-arrestin assay.

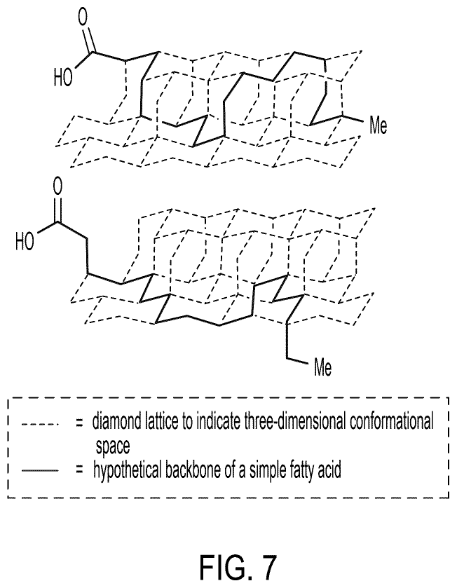

The present method can also comprise creating a model that shows conformation of the target fatty acid bound to the fatty acid receptor. The model is based on the assessment of which of the mimetics of the set induced the desired function of the fatty acid receptor. For example, the model can comprise a lattice that depicts the three-dimensional space of the target fatty acid bound to the fatty acid receptor. An exemplary lattice that depicts two potential three-dimensional conformations of oleic acid is shown in FIG. 7.

### Example 1

In this example, four different classes of oleic acid mimetics (termed Class I-IV) were prepared using the synthetic schemes set forth herein. Each class differs by the position of the conformational biasing element in the C18 backbone. The carbon number where each conformational constraint begins within each ligand class is specified. The set of compounds is depicted in Table 2 and includes six different compounds for each mimetic “Class” (1A-F, 2A-F, 3A-F, and 4A-F).

TABLE 2
Class I:

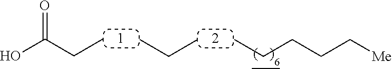

| | |
|---|---|
| 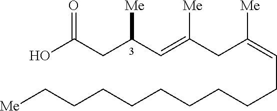 [SMILES: CCCCCCCCCC/C=C(/C)C/C(C)=C/[C@H](C)CC(=O)O] | 1-A |
| 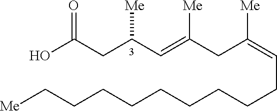 [SMILES: CCCCCCCCCC/C=C(/C)C/C(C)=C/[C@@H](C)CC(=O)O] | 1-B |
| 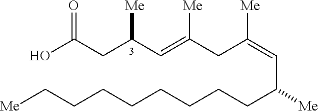 [SMILES: CCCCCCCCC[C@@H](C)/C=C(/C)C/C(C)=C/[C@H](C)CC(=O)O] | 1-C |
| 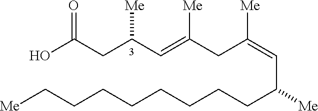 [SMILES: CCCCCCCCC[C@@H](C)/C=C(/C)C/C(C)=C/[C@@H](C)CC(=O)O] | 1-D |
| 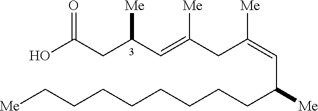 [SMILES: CCCCCCCCC[C@H](C)/C=C(/C)C/C(C)=C/[C@H](C)CC(=O)O] | 1-E |
| 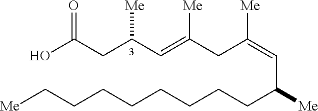 [SMILES: CCCCCCCCC[C@H](C)/C=C(/C)C/C(C)=C/[C@@H](C)CC(=O)O] | 1-F |

Class II:

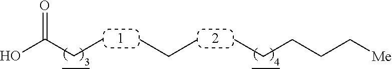

| | |
|---|---|
| 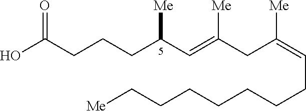 [SMILES: CCCCCCCC/C=C(/C)C/C(C)=C/[C@H](C)CCCC(=O)O] | 2-A |
| 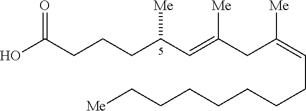 [SMILES: CCCCCCCC/C=C(/C)C/C(C)=C/[C@@H](C)CCCC(=O)O] | 2-B |
| 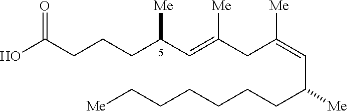 [SMILES: CCCCCCC[C@@H](C)/C=C(/C)C/C(C)=C/[C@H](C)CCCC(=O)O] | 2-C |
| 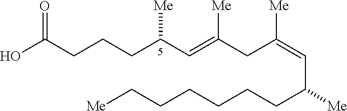 [SMILES: CCCCCCC[C@@H](C)/C=C(/C)C/C(C)=C/[C@@H](C)CCCC(=O)O] | 2-D |
| 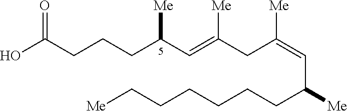 [SMILES: CCCCCCC[C@H](C)/C=C(/C)C/C(C)=C/[C@H](C)CCCC(=O)O] | 2-E |
| 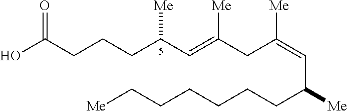 [SMILES: CCCCCCC[C@H](C)/C=C(/C)C/C(C)=C/[C@@H](C)CCCC(=O)O] | 2-F |

Class III:

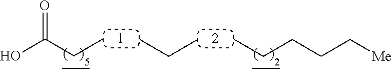

| | |
|---|---|
| 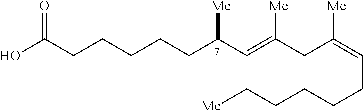 [SMILES: CCCCCC/C=C(/C)C/C(C)=C/[C@H](C)CCCCCC(=O)O] | 3-A |
| 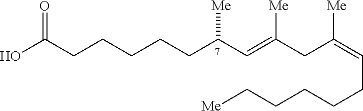 [SMILES: CCCCCC/C=C(/C)C/C(C)=C/[C@@H](C)CCCCCC(=O)O] | 3-B |
| 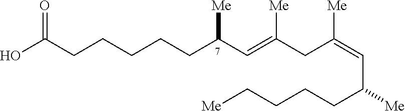 [SMILES: CCCCC[C@@H](C)/C=C(/C)C/C(C)=C/[C@H](C)CCCCCC(=O)O] | 3-C |
| 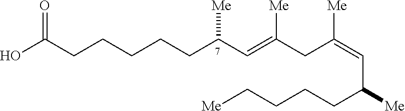 [SMILES: CCCCC[C@H](C)/C=C(/C)C/C(C)=C/[C@@H](C)CCCCCC(=O)O] | 3-D |
| 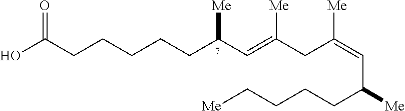 [SMILES: CCCCC[C@H](C)/C=C(/C)C/C(C)=C/[C@H](C)CCCCCC(=O)O] | 3-E |
| 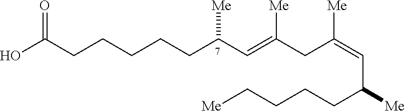 [SMILES: CCCCC[C@H](C)/C=C(/C)C/C(C)=C/[C@@H](C)CCCCCC(=O)O] | 3-F |

Class IV:

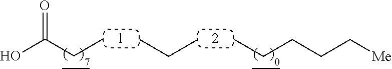

| | |
|---|---|
| 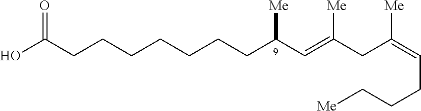 [SMILES: CCCC/C=C(/C)C/C(C)=C/[C@H](C)CCCCCCCC(=O)O] | 4-A |
| 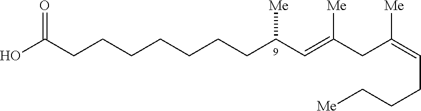 [SMILES: CCCC/C=C(/C)C/C(C)=C/[C@@H](C)CCCCCCCC(=O)O] | 4-B |
| 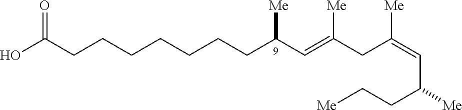 [SMILES: CCC[C@@H](C)/C=C(/C)C/C(C)=C/[C@H](C)CCCCCCCC(=O)O] | 4-C |
| 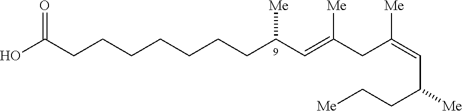 [SMILES: CCC[C@@H](C)/C=C(/C)C/C(C)=C/[C@@H](C)CCCCCCCC(=O)O] | 4-D |
| 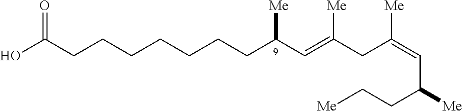 [SMILES: CCC[C@H](C)/C=C(/C)C/C(C)=C/[C@H](C)CCCCCCCC(=O)O] | 4-E |
| 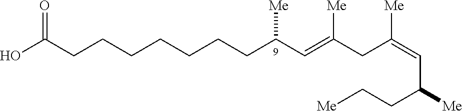 [SMILES: CCC[C@H](C)/C=C(/C)C/C(C)=C/[C@@H](C)CCCCCCCC(=O)O] | 4-F |

For Class 1 compounds, the chiral conformational constraint elements first appear at C3 of the fatty acid backbone, with the other classes moving the positioning of the constraints to begin at C5, C7 and C9, respectively. Comparing the compounds across each Glass, ligands with the same alphabetical descriptor (e.g., “A”, vs “B”, vs “C”, etc. . . . ) share the same stereodefined constraint (same substitution and stereochemistry), but differ with respect to where the constraint is placed within the 18-carbon backbone.

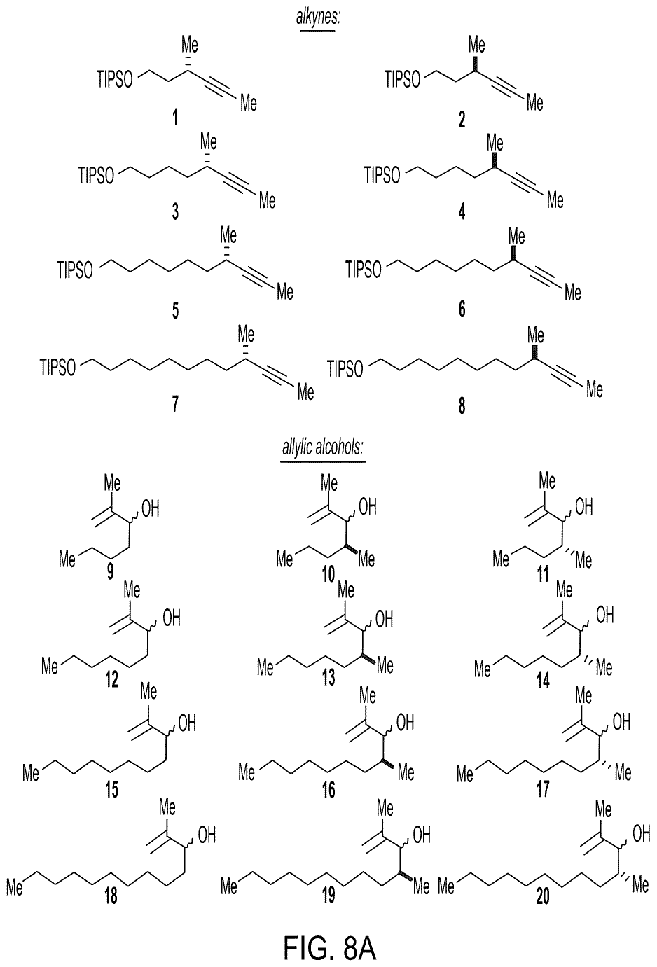

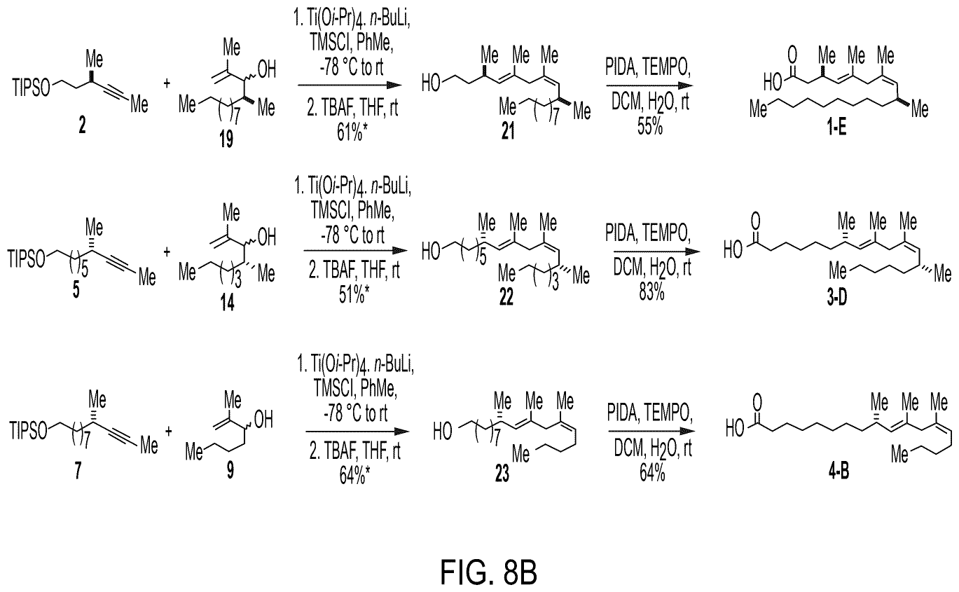

FIGS. 8A and 8B illustrate the convergent synthesis of members of the fatty acid mimetic collection. FIG. 8A shows structures of the alkynes and allylic alcohols employed.

FIG. 8B provides examples of the chemical pathway for synthesis of members of the fatty acid mimetic collection. The three-step process enabled stereoselective coupling and conversion to the designed fatty acid mimetics.

### Example 2

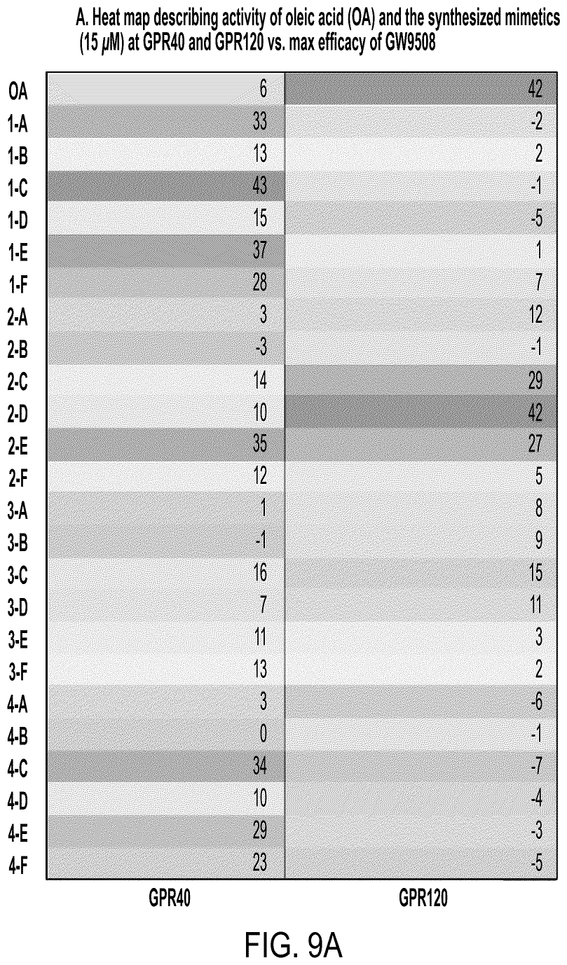

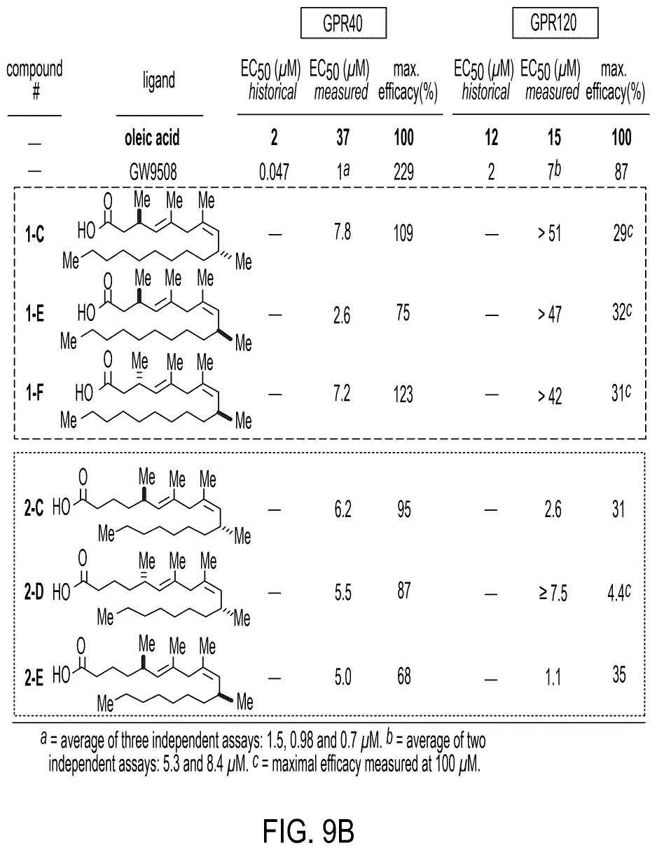

In this example, compounds from Example 1 were assessed as functional ligands (agonists) to two oleic acid receptors (GPR40 and GPR120). Notably, the structure of GPR40 bound to oleic acid is not known, while structural information regarding the GPR120-oleic acid complex is only beginning to emerge. The experiments were conducted at a single concentration (15 μM) in a commercial β-arrestin assay for GPR40 and GPR120. The results from these experiments are depicted as a heat map in FIG. 9A and are compared to the observed activity of oleic acid at 15 μM dosing (“OA”).

As can be seen from this heat map, the collection of oleic acid mimetics in the “1-X” group appear enriched for activity at GPR40 (e.g. 1-A, 1-C, 1-E), and this subset is distinct from the group that are enriched for activity at GPR120 are distinct from those that appear optimal for GPR120 (2-C, 2-D, and 2-E).

### Example 3

In this example, selected compounds from Example 1 (Compounds 1-C, 1-E, 1-F, 2-C, 2-D and 2-E) were evaluated in dose-response experiments that provide an indication of their potency and efficacy. The compounds were compared with the known ligand oleic acid and a synthetic dual GPR40 and GPR120 agonist well known to those skilled in the art (“GW9508”). Notably, oleic acid has been reported to have potent stimulatory activity on CHO-mGPR40 cells (EC₅₀=2 μM) and induces insulin secretion in MIN6 cells at concentrations as low as 1 μM while also being an agonist of GPR120 with an EC₅₀ of 12 μM. The evaluation of the selected compounds, oleic acid and GW9508 was conducted with a PathHunter® β-arrestin assay that monitors the activation of a GPCR in a homogenous, non-imaging assay format using Enzyme Fragment Complementation (EFC) with β-galactosidase as the functional reporter. Importantly, in this particular assay, oleic acid has an EC₅₀ of 37 μM at GPR40, and 15 μM at GPR120, while GW9508 has an EC₅₀ of 1 μM at GPR40 and 7 μM at GPR120.

FIG. 9B provides data from the experiments, including a comparison of EC₅₀ values for the most potent compounds at GPR40 and GPR120.

Apparently, this reporter assay delivers results for these positive controls that are right shifted by ~20-fold for both oleic acid and GW9508. Fatty acid mimetics 1-C, 1-E, and 1-F, proved to be exceptionally selective and potent agonists of GPR40. These compounds activate GPR40 with similar efficacy to oleic acid (76 and 124%), doing so with substantial selectivity over GPR120 and potency up to twelve times greater than oleic acid (i.e., 1-E). Unlike these fatty acid mimetics from “Class 1”, the “Class 2” hits proved to have a distinct selectivity profile. As illustrated in FIG. 9B, compounds 2-C, 2-D, and 2-E were effective agonists of GPR40 with approximately 6× the potency of oleic acid and similar efficacy (69 to 96%), but they were also agonists of GPR120 with EC₅₀ values from 1 to ~7 μM, albeit displaying agonist efficacy at a fraction of what is observed with oleic acid and GW9508. Notably, fatty acid mimetic 2-E has a potency and selectivity profile that was not observed with oleic acid or GW9508, showing 5-fold selectivity for GPR120 and a 7-15-fold enhanced potency.

### Example 4

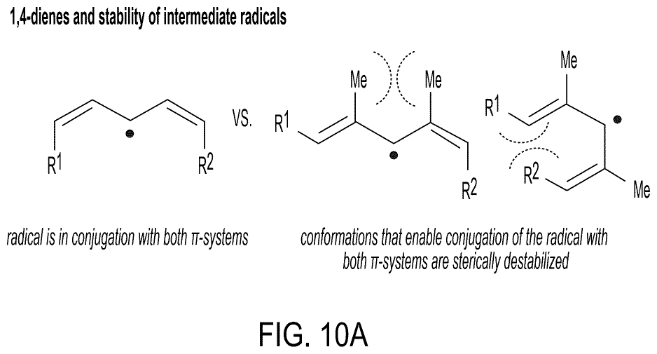

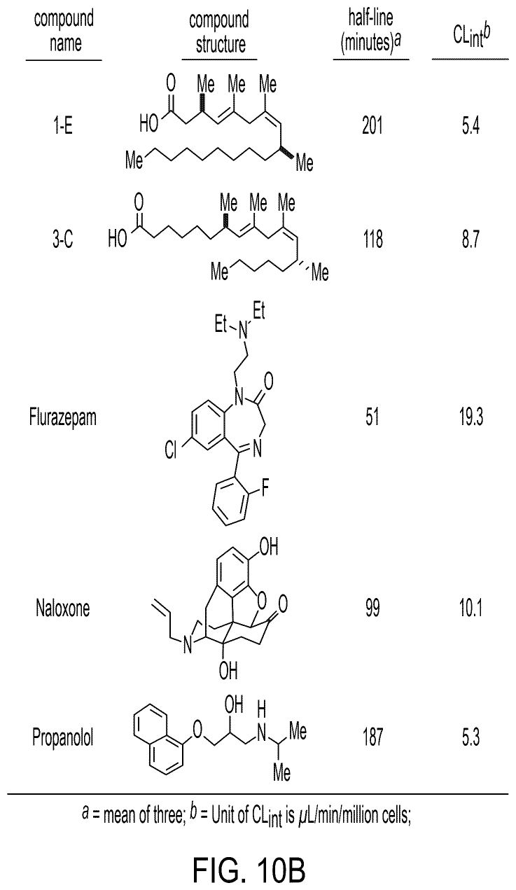

In this example, the metabolic stability of some of the compounds prepared in Example 1 were evaluated. The stereodefined 1,4-diene of the compounds had substitution not typically seen in natural fatty acids (both alkenes are trisubstituted). It was hypothesized that this molecular feature would impart added stability to these fatty acid mimetics in comparison to typical polyunsaturated fatty acids comprising skipped polyenes, as the central sp³ carbon of the 1,4-dienes present would be less likely to participate in hydrogen atom abstraction chemistry because the resulting pentadienyl radical would not easily be stabilized by both alkenes (as illustrated in FIG. 10A).

An investigation of in vitro metabolism was performed by assessing the intrinsic clearance of two of the fatty acid mimetics in human hepatocytes. As depicted in FIG. 10B compounds 1-E and 3-C were found to have half-lives of 201 and 118 minutes, respectively, both being substantially greater than Flurazepan and being similar to Naloxone and Propanolol controls.

The CLᵢₙₜ values observed for 1-E and 3-C of 5.4 and 8.7 μL/min/million cells respectively would indicate a low turnover of the carboxylic acids assessed in the in vitro system.

The unique potency and selectivity profiles of the chiral conformationally constrained compounds synthesized and identified in the foregoing Examples supports the conclusion that fatty acid mimetics (such as the disclosed mimetics of oleic acid) prepared by metallacycle-mediated coupling of alkynes with allylic alcohols are unique natural product-inspired functional agonists of fatty acid receptors (such as GPR40 and GPR120).

### Example 5

In this example, selective conformational bias of the ligands was assessed using the reported structure of GPR120 bound to oleic acid (PDB: 8id6). Mao, et al., Unsaturated bond recognition leads to biased signal in a fatty acid receptor. Science, 2023, 380, eadd6220.

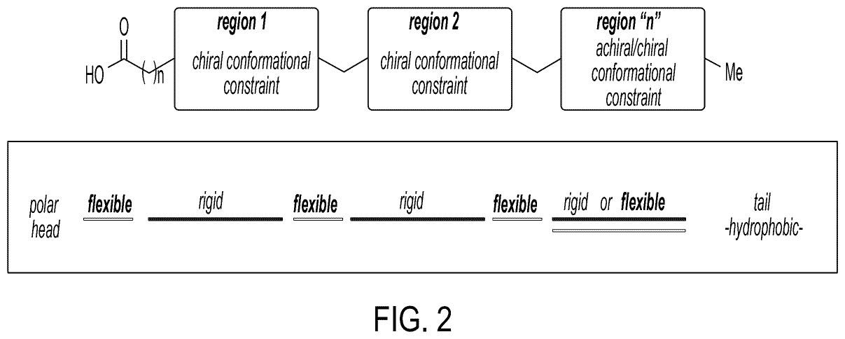

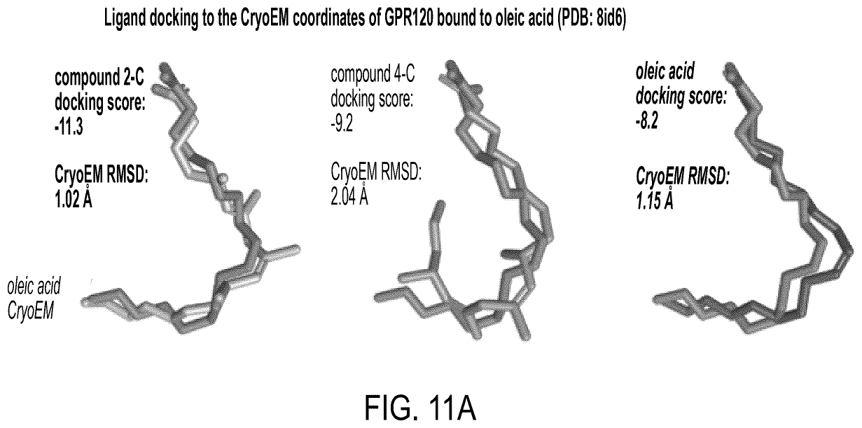

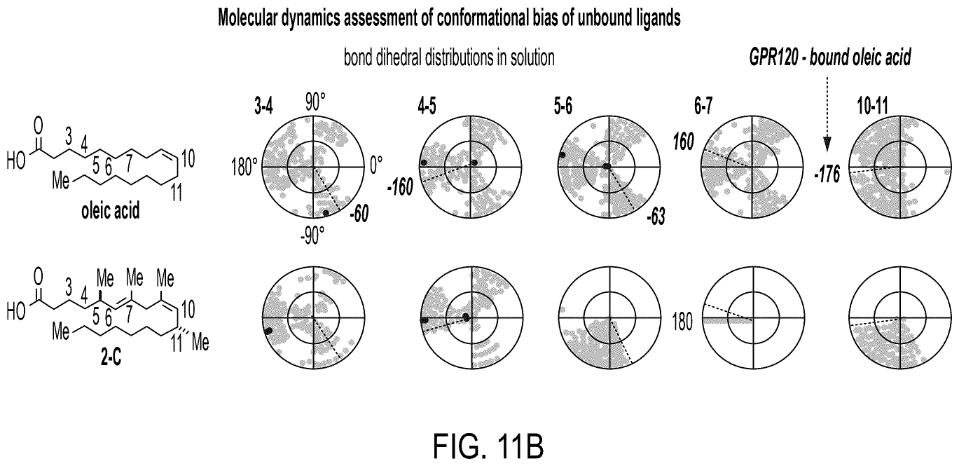

Molecular docking to the GPR120 coordinates showed that 2-C, 2-E and oleic acid readily adopt a binding mode and aliphatic chain conformation which deviates minimally from the oleic acid molecule modelled in the Cryo-EM structure. Conversely, 4-C that was found to be inactive against GPR120 (FIG. 8A) exhibited a marked deviation from the experimental oleic acid molecule, especially in the conformation associated with the region spanning carbons 14-18 (FIG. 11A). To further investigate whether the improved docking parameters for GPR120-active compounds relative to an inactive compound arose from a differential conformational bias between these molecules in their unbound state, we performed molecular dynamics (MD) simulations of the unbound molecules in water. Inspection of bond dihedral angle distributions over the course of the MD trajectories of 2-C (FIG. 11B) and 2-E showed a preference for subsets of dihedral angles near the angles observed in the GPR120-bound conformation of oleic acid at the bonds between C3-C4, C4-C5, C5-C6, and C10-C11 compared to the MD trajectory of unbound oleic acid (FIG. 10B). Conversely, 4-C showed a conformational bias against the dihedral angles observed in the GPR120-bound conformation of oleic acid at the bonds between carbons spanning C13-C16 and 9-10.

The various compounds, materials, structures, methods, and systems are included by way of illustration and example only and not in any limiting sense. In view of this disclosure, the present teachings may be implemented in other applications and approaches, while remaining within the scope of the appended claims. Variations and modifications may be made to the embodiments described herein without substantially departing from the spirit and principles of the teachings described herein.

### Exemplary Embodiments

Exemplary Embodiment 1. A compound of Formula (I):

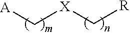

or a pharmaceutically acceptable salt thereof; wherein:
A comprises a polar head group;
X comprises a conformationally constrained core comprising two or more conformational constraints and at least one optional flexible subunit;
R comprises a non-polar group; and
each of m and n is independently an integer from 1 to 25, or from 1 to 19, or from 1 to 11, or from 1 to 7.

Exemplary Embodiment 2. The compound or pharmaceutically acceptable salt of exemplary embodiment 1, wherein the flexible subunit is a methylene subunit.

Exemplary Embodiment 3. The compound or pharmaceutically acceptable salt of exemplary embodiment 1, wherein X comprises (ix)

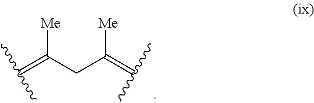 [SMILES: *=C(C)CC(=*)C]

Exemplary Embodiment 4. The compound or pharmaceutically acceptable salt of exemplary embodiment 1, wherein each the two or more conformational constraints is independently selected from the group consisting of (x), (x′), (x″), and (x′″):

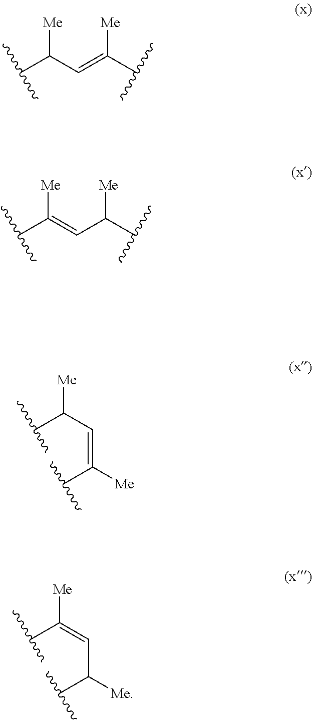 [SMILES: */C(C)=C/C(*)C ; */C(C)=C/C(*)C ; */C(C)=C\C(*)C ; */C(C)=C\C(*)C]

Exemplary Embodiment 5. The compound or pharmaceutically acceptable salt of exemplary embodiment 4, wherein the two or more conformational constraints are joined by a C₁₋₆-alkylene, such as a C₁₋₃-alkylene (e.g., methylene, ethylene, or propylene), preferably C₁ (e.g., methylene).

Exemplary Embodiment 6. The compound or salt of any of exemplary embodiments 1 to 5, wherein A comprises A²⁻R¹⁵⁻, wherein A² is a polar head group, and R¹⁵ is a bond, a C₁₋₆-alkyl, or a C₂-C₆ alkenyl.

Exemplary Embodiment 7. The compound or salt of exemplary embodiment 6, wherein A² is a carboxylic acid, a carboxyl ester, an amide, a ketone, a phosphonic acid, a phosphinic acid, a sulfonic acid, a sulfinic acid, a sulfonamide, an acyl-sulfonamide, a hydroxamic acid, a hydroxamic ester, a sulfonylurea, an acylurea, a tetrazole, a thiazolidinedione, an oxazolidinedione, an oxadiazolone, a thiadiazolone, an oxathiadiazole oxide, an oxadiazolethione, an isoxazole, a tetramic acid, a cyclopentane diones, a phenol derivative, a squaric acid derivative, or a salt thereof.

Exemplary Embodiment 8. A compound of Formula (II):

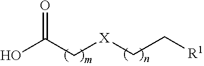

or a pharmaceutically acceptable salt thereof; wherein:
X comprises a constrained core comprising one or more acyclic conformational constraints and at least one optional flexible subunit;
R¹ is a C₁₋₆-alkyl; and
each of m and n is independently an integer from 1 to 25, or from 1 to 19, or from 1 to 11, or from 1 to 7;
provided that the compound is not a natural product and/or
provided that at least one conformational constraint is a conformational constraint other than

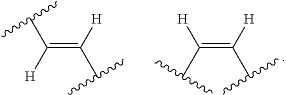

Exemplary Embodiment 9. The compound or pharmaceutically acceptable salt of exemplary embodiment 8, wherein the constrained core comprises two or more acyclic conformational constraints.

Exemplary Embodiment 10. The compound or pharmaceutically acceptable salt of exemplary embodiment 8 or claim 9, wherein each of the one or more acyclic conformational constraints is (x), (x′), (x″), or (x′″):

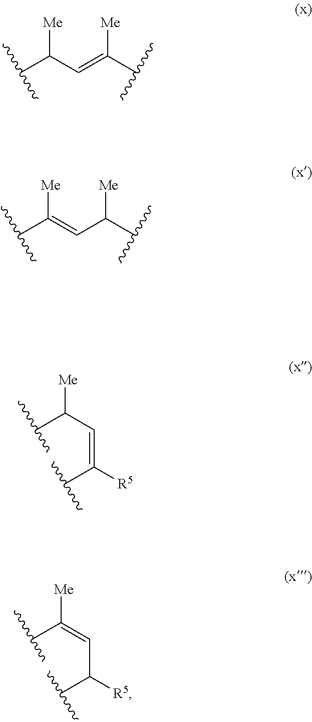

wherein R⁵ is hydrogen or C₁₋₆-alkyl.

Exemplary Embodiment 11. The compound or pharmaceutically acceptable salt of any one of exemplary embodiments 8-10, wherein each acyclic conformational constraint is joined by a C₁₋₆-alkylene, such as a C₁₋₃-alkylene (e.g., methylene, ethylene, or propylene), preferably C1 (e.g., methylene).

Exemplary Embodiment 12. The compound or salt of any one of exemplary embodiments 8-10, wherein X comprises:

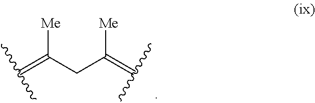 [SMILES: *=C(C)CC(=*)C]

Exemplary Embodiment 13. The compound or pharmaceutically acceptable salt of exemplary embodiment 8, wherein X is:

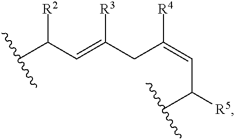

wherein:
R² is C₁₋₆-alkyl;
R³ is C₁₋₆-alkyl;
R⁴ is C₁₋₆-alkyl; and
R⁵ is hydrogen or C₁₋₆-alkyl.

Exemplary Embodiment 14. A compound of Formula (III):

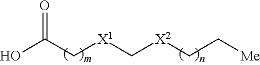

or a pharmaceutically acceptable salt thereof; wherein:
X¹ is

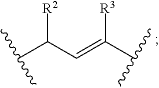

R² is C₁₋₆-alkyl;
R³ is C₁₋₆-alkyl;
X² is

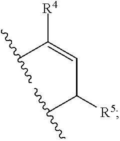

R⁴ is C₁₋₆-alkyl;
R⁵ is hydrogen, deuterium, or C₁₋₆-alkyl; and
each of m and n is independently an integer from 1 to 25, or from 1 to 19, or from 1 to 11, or from 1 to 7.

Exemplary Embodiment 15. The compound or pharmaceutically acceptable salt of exemplary embodiment 14, wherein
X¹ is (x.1) or (x.2):

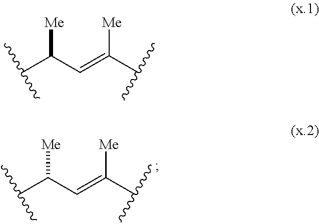 [SMILES: */C(C)=C/[C@@H](*)C ; */C(C)=C/[C@H](*)C]

and
X² is (x′″.1) or (x′″.2):

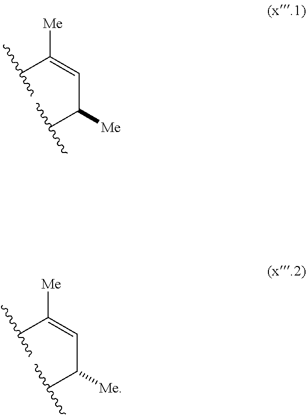 [SMILES: */C(C)=C\[C@H](*)C ; */C(C)=C\[C@@H](*)C]

Exemplary Embodiment 16. The compound or pharmaceutically acceptable salt of exemplary embodiment 13 or claim 14, wherein each of R², R³, and R⁴ are methyl or a partially or fully deuterated methyl, and R⁵ is hydrogen, deuterium, methyl, or a partially or fully deuterated methyl.

Exemplary Embodiment 17. The compound or pharmaceutically acceptable salt of exemplary embodiment 13 or claim 14, wherein each of R², R³, and R⁴ are methyl or a partially or fully deuterated methyl, and R⁵ is hydrogen or deuterium.

Exemplary Embodiment 18. The compound or pharmaceutically acceptable salt of exemplary embodiment 13 or claim 14, wherein each of R², R³, and R⁴ are methyl or a partially or fully deuterated methyl, and R⁵ is C₁₋₆-alkyl.

Exemplary Embodiment 19. The compound or pharmaceutically acceptable salt of exemplary embodiment 13 or claim 14, wherein each of R², R³, R⁴, and R⁵ are methyl or a partially or fully deuterated methyl.

Exemplary Embodiment 20. The compound or pharmaceutically acceptable salt of any one of the preceding claims, wherein m is 1, 3, 5, 7, 9, 11, 13, 15, 17, 19, 21, 23 or 25.

Exemplary Embodiment 21. The compound or pharmaceutically acceptable salt of any one of the preceding claims, wherein n is 1, 3, 5, 7, 9, 11, 13, 15, 17, 19, 21, 23 or 25.

Exemplary Embodiment 22. The compound or pharmaceutically acceptable salt of any one of the preceding claims, wherein m+n is 6, 7, 8, 9, 10, 11, 12, 13, 14, 15, 16, 17, 18, 19, 20, 21, 22, 23, 24 25, 26, 27, 28, 29, 30, 31, 32, 33, 34, 35, 36, 37, 38, 39, or 40.

Exemplary Embodiment 23. A compound or a pharmaceutically acceptable salt thereof, wherein the compound is selected from one of Compounds 1-A to 4-F:

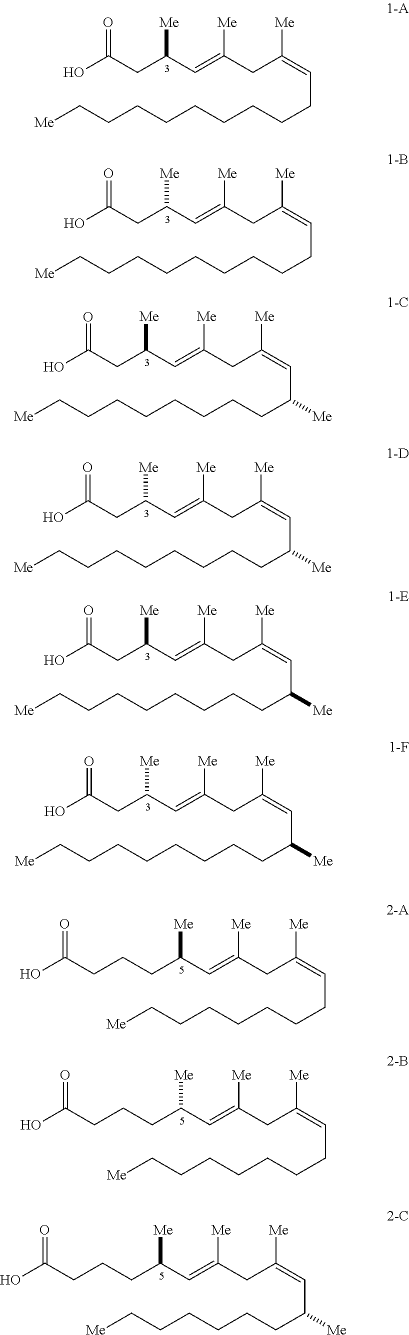

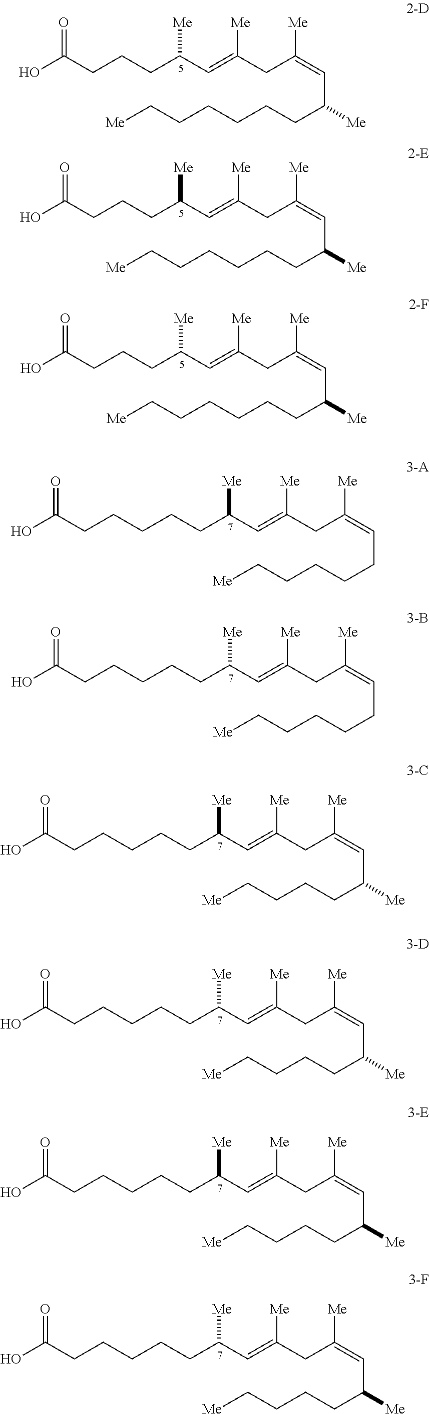

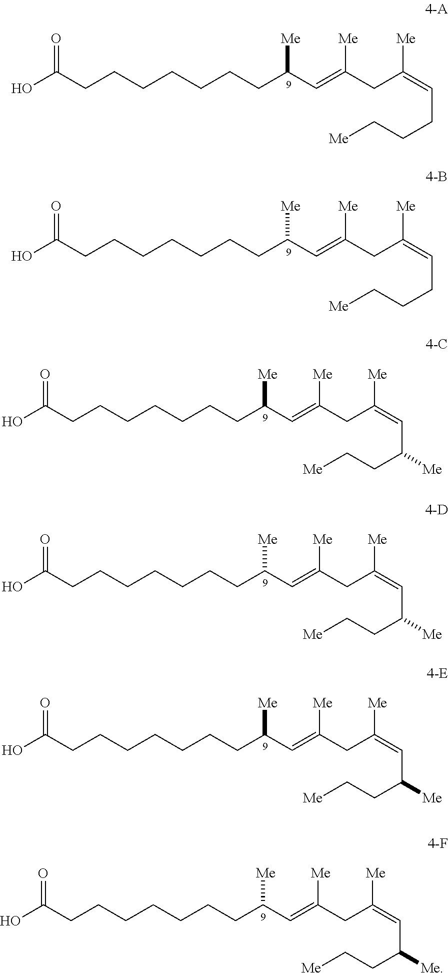 [SMILES: CCCC/C=C(/C)C/C(C)=C/[C@H](C)CCCCCCCC(=O)O ; CCCC/C=C(/C)C/C(C)=C/[C@@H](C)CCCCCCCC(=O)O ; CCC[C@@H](C)/C=C(/C)C/C(C)=C/[C@H](C)CCCCCCCC(=O)O ; CCC[C@@H](C)/C=C(/C)C/C(C)=C/[C@@H](C)CCCCCCCC(=O)O ; CCC[C@H](C)/C=C(/C)C/C(C)=C/[C@H](C)CCCCCCCC(=O)O ; CCC[C@H](C)/C=C(/C)C/C(C)=C/[C@@H](C)CCCCCCCC(=O)O]

Exemplary Embodiment 24. A method for modulating activity of GPR40 and/or GPR120, the method comprising contacting GPR40 and/or GPR120 with the compound or the pharmaceutically acceptable salt of any one of exemplary embodiments 1-23.

Exemplary Embodiment 25. A method for treating a metabolic disease (e.g., type 2 diabetes) in a subject in need thereof, the method comprising administering to the subject the compound or the pharmaceutically acceptable salt of any one of any one of exemplary embodiments 1-23.

Exemplary Embodiment 26. A method for treating obesity in a subject in need thereof, the method comprising administering to the subject the compound or the pharmaceutically acceptable salt of any one of any one of exemplary embodiments 1-23.

Exemplary Embodiment 27. A kit or set comprising two or more fatty acid mimetics, wherein each of the fatty acid mimetics is: a compound of formula (I), (II), or (III) or a pharmaceutically acceptable salt thereof.

Exemplary Embodiment 28. The kit or set of exemplary embodiment 27, wherein each of the fatty and acid mimetics is in a separate vessel.

Exemplary Embodiment 29. The kit or set of exemplary embodiment 27 or 28, wherein each of the mimetics has a constrained conformation, and
each of the mimetics in the set has a different structure from each of the other mimetics in the set.

Exemplary Embodiment 30. The kit or set of an one of exemplary embodiments 27-29, comprising at least 4, 6, 12 or 24 of said fatty acid mimetics.

Exemplary Embodiment 31. The kit or set of any one of exemplary embodiments 27-29, wherein each of the fatty acid mimetics is a mimetic of:
(a) a C12 fatty acid;
(b) a C14 fatty acid;
(c) a C16 fatty acid;
(d) a C18 fatty acid;
(e) a C20 fatty acid; or
(f) a C22 fatty acid.

Exemplary Embodiment 32. The kit or set of any one of exemplary embodiments 27-29, comprising each of Compounds 1-A to 4-F in separate vessels.

Exemplary Embodiment 33. A method for making the compound or salt of any one of exemplary embodiments 1-23, wherein the method is based on the retrosynthetic strategy shown in FIG. 6B.

Exemplary Embodiment 34. A method for making a compound of Formula (I), the method comprising:
coupling an alkyne of Formula (IV):

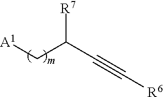

wherein A¹ comprises a polar head group, or a head group protected with a protecting group;
R⁶ is a C₁₋₆-alkyl;
R⁷ is hydrogen or a C₁₋₆-alkyl;
with an allylic alcohol of Formula (V):

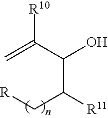

wherein R comprises a non-polar group;
R¹⁰ is hydrogen or a C₁₋₆-alkyl;
R¹¹ is hydrogen or a C₁₋₆-alkyl; and
each of m and n is independently an integer from 1 to 25, or from 1 to 19, or from 1 to 11, or from 1 to 7.

Exemplary Embodiment 35. The method of exemplary embodiment 34, wherein the coupling is metallacycle-mediated coupling.

Exemplary Embodiment 36. The method of exemplary embodiment 35, wherein the metallacycle-mediated coupling comprises coupling the alkyne and the allylic alcohol in the presence of a titanium alkoxide, such as Ti(Oi-Pr)₄, and an organolithum, such as n-butyl lithium (n-BuLi).

Exemplary Embodiment 37. The method of exemplary embodiment 36, wherein the metallacycle-mediated coupling further comprises coupling the alkyne and the allylic alcohol in the presence of a silylating agent, such as trimethylsilyl chloride (TMSCl).

Exemplary Embodiment 38. The method of exemplary embodiment 34, wherein the alkyne has a structure of Formula (IV-A):

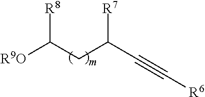

wherein
R^{a} is hydrogen or a C₁₋₆-alkyl; and
R⁹ is hydrogen or an oxygen protecting group.

Exemplary Embodiment 39. The method of exemplary embodiment 38, wherein the compound has a structure of Formula (IV-A-1):

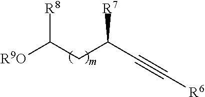

wherein R⁷ is a C₁₋₆-alkyl.

Exemplary Embodiment 40. The method of exemplary embodiment 38, wherein the compound has a structure of Formula (IV-A-2):

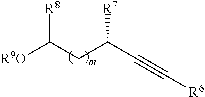

wherein R⁷ is a C₁₋₆-alkyl.

Exemplary Embodiment 41. The method of any of exemplary embodiments 38-40, wherein R⁸ is hydrogen; and R⁹ is an oxygen protecting group.

Exemplary Embodiment 42. The method of exemplary embodiment 41, wherein R⁹ is an oxygen protecting group selected from methyl, benzyl, p-methoxybenzyl, methoxymethyl (MOM), tert-butyldimethylsilyl (TBS), tert-butyldiphenylsilyl (TBDPS), and triisopropylsilyl (TIPS).

Exemplary Embodiment 43. The method of exemplary embodiment 34, further comprising making the alkyne by carbonyl alkynylation of a compound of Formula VI:

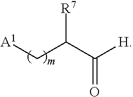

Exemplary Embodiment 44. The method of exemplary embodiment 38, further comprising making the alkyne by carbonyl alkynylation of a compound of Formula VI-A:

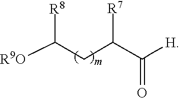

Exemplary Embodiment 45. The method of exemplary embodiment 34, wherein the allylic alcohol has a structure of Formula (V-A):

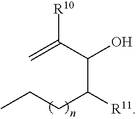

Exemplary Embodiment 46. The method of exemplary embodiment 45, wherein the allylic alcohol has a structure of Formula (V-A-1):

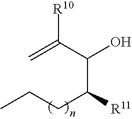

wherein R¹¹ is a C₁₋₆-alkyl.

Exemplary Embodiment 47. The method of exemplary embodiment 45, wherein the allylic alcohol has a structure of Formula (V-A-2):

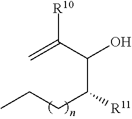

wherein R¹¹ is a C₁₋₆-alkyl.

Exemplary Embodiment 48. The method of exemplary embodiment 34, further comprising making the allylic alcohol by Grignard reaction of (R¹⁰)(CH₂)CH—MgR¹² with a compound of Formula (VII):

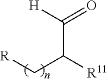

wherein R¹² is a halogen.

Exemplary Embodiment 49. The method of exemplary embodiment 45, further comprising making the allylic alcohol by Grignard reaction of (R¹⁰)(CH₂)C—MgR¹² with a compound of Formula (VII-A):

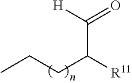

wherein R¹² is a halogen.

Exemplary Embodiment 50. The method of any one of exemplary embodiments 43, 44, 48 or 49, further comprising making the compound of any of Formulas (VI, VI-A, VII, or VII-A) by stereoselective alkylation of a compound of Formula (VIII):

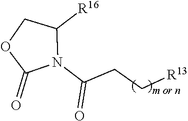

wherein R¹³ is C₁₋₆-alkyl or A¹; and
R¹⁶ is a hydrogen, alkyl, aryl or arylalkyl.

Exemplary Embodiment 51. The method of exemplary embodiment 34, further comprising:
a) stereoselectively alkylating a compound of Formula (VIII):

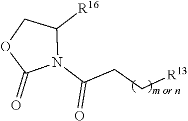

wherein R¹³ is C₁₋₆-alkyl, and R¹⁶ is a hydrogen, alkyl, aryl or arylalkyl, to form a compound of Formula (VII):

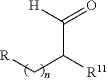

b) reacting the compound of Formula (VII) to form the allylic alcohol of Formula (V);
c) stereoselectively alkylating a compound of Formula (VIII):

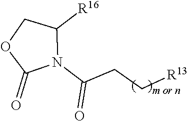

wherein R¹³ is A¹, and R¹⁶ is a hydrogen, alkyl, aryl or arylalkyl, to form a compound of Formula (VI):

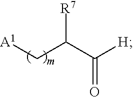

and
d) making the alkyne of Formula (IV) by carbonyl alkynylation of the compound of Formula (VI).

Exemplary Embodiment 52. The method of exemplary embodiment 34 or 51, wherein A¹ comprises a head group protected with a protecting group, and the method further comprises:
deprotecting and oxidizing the head group to form a carboxylic acid group.

Exemplary Embodiment 53. The method of exemplary embodiment 51, wherein step b) comprises
a reaction with a Grignard reagent,
a reaction with a vinyl lithium reagent,
catalytic addition by NHK coupling, or
reaction of an organolanthanum or organocerium reagent to an aldehyde.

Exemplary Embodiment 54. A method of conformational profiling comprising:
preparing a set of mimetics of the target fatty acid; and
assessing the functional activity of each member of the set of fatty acid mimetics at a desired fatty acid receptor (e.g., GPR40).

Exemplary Embodiment 55. The method of exemplary embodiment 54, further comprising identifying one or more subpopulations of the set that have structural features that are preferred for achieving the desired function.

Exemplary Embodiment 56. The method of exemplary embodiment 55, further comprising preparing a subset of fatty acid mimetics comprising the structural features of the one or more subpopulations, wherein the mimetics of the subset comprise additional structural rigidification compared to the mimetics of the subpopulation.

Exemplary Embodiment 57. The method of exemplary embodiment 54, wherein the mimetics of the set have at least 6 different structures.

Exemplary Embodiment 58. The method of exemplary embodiment 54, wherein the mimetics of the set have at least 24 different structures.

Exemplary Embodiment 59. The method of any one of exemplary embodiments 54-58, wherein each of the fatty acid mimetics is a mimetic of:
(a) a C12 fatty acid;
(b) a C14 fatty acid;
(c) a C16 fatty acid;
(d) a C18 fatty acid;
(e) a C20 fatty acid; or
(f) a C22 fatty acid.

Exemplary Embodiment 60. The method of any one of exemplary embodiments 54-59, wherein each of the mimetics of the set is a C18 fatty acid mimetic, and the set comprises each of Compounds 1-A to 4-F:

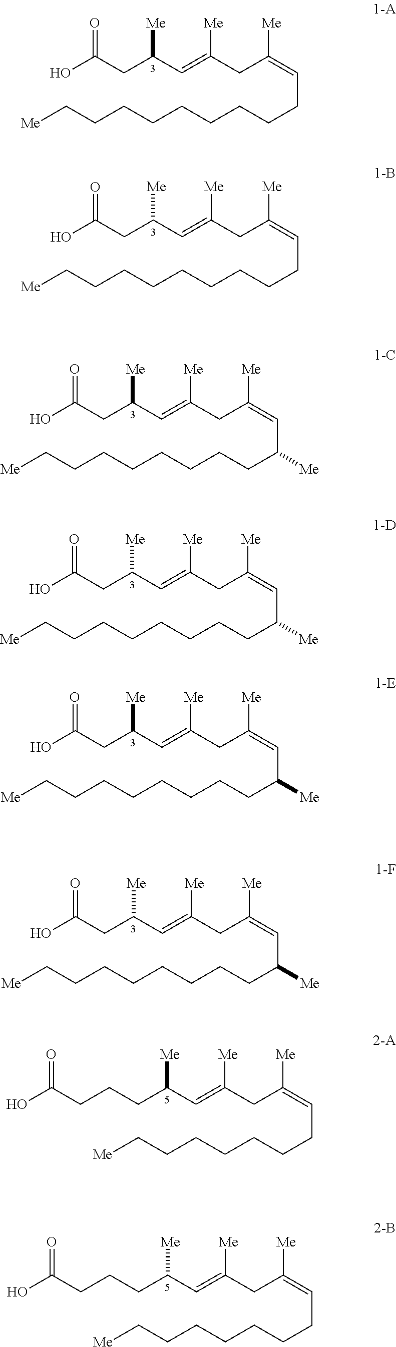

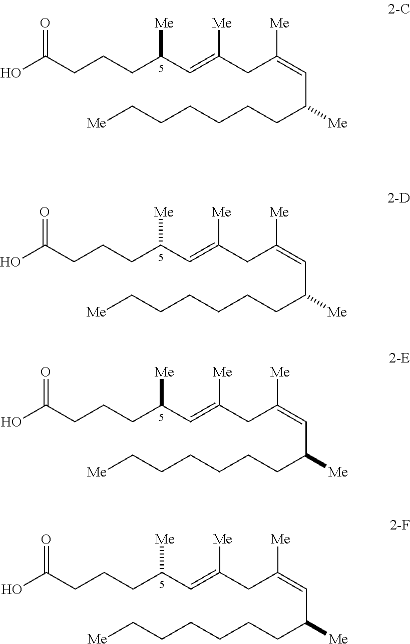 [SMILES: CCCCCCC[C@@H](C)/C=C(/C)C/C(C)=C/[C@H](C)CCCC(=O)O ; CCCCCCC[C@@H](C)/C=C(/C)C/C(C)=C/[C@@H](C)CCCC(=O)O ; CCCCCCC[C@H](C)/C=C(/C)C/C(C)=C/[C@H](C)CCCC(=O)O ; CCCCCCC[C@H](C)/C=C(/C)C/C(C)=C/[C@@H](C)CCCC(=O)O]

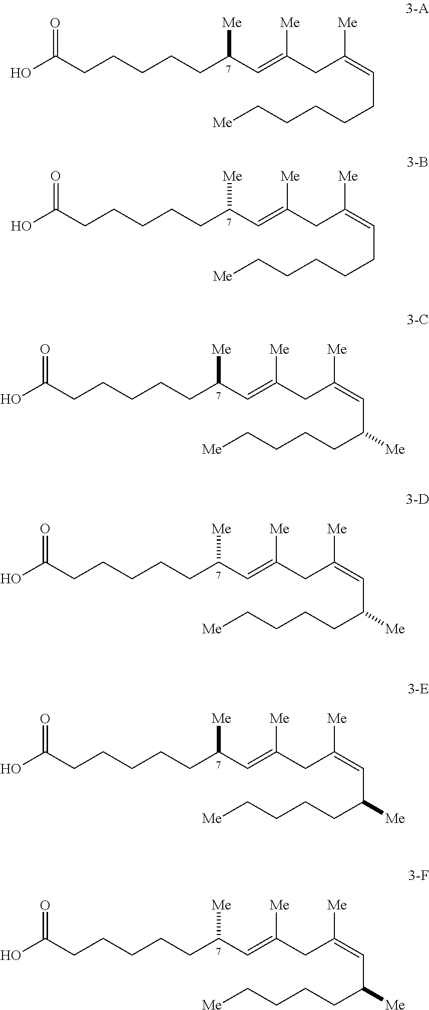 [SMILES: CCCCCC/C=C(/C)C/C(C)=C/[C@H](C)CCCCCC(=O)O ; CCCCCC/C=C(/C)C/C(C)=C/[C@@H](C)CCCCCC(=O)O ; CCCCC[C@@H](C)/C=C(/C)C/C(C)=C/[C@H](C)CCCCCC(=O)O ; CCCCC[C@@H](C)/C=C(/C)C/C(C)=C/[C@@H](C)CCCCCC(=O)O ; CCCCC[C@H](C)/C=C(/C)C/C(C)=C/[C@H](C)CCCCCC(=O)O ; CCCCC[C@H](C)/C=C(/C)C/C(C)=C/[C@@H](C)CCCCCC(=O)O]

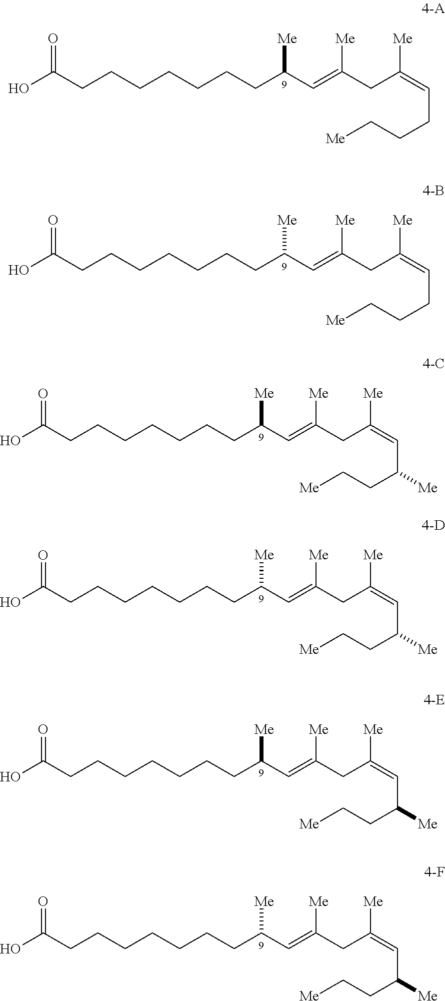 [SMILES: CCCC/C=C(/C)C/C(C)=C/[C@H](C)CCCCCCCC(=O)O ; CCCC/C=C(/C)C/C(C)=C/[C@@H](C)CCCCCCCC(=O)O ; CCC[C@@H](C)/C=C(/C)C/C(C)=C/[C@H](C)CCCCCCCC(=O)O ; CCC[C@@H](C)/C=C(/C)C/C(C)=C/[C@@H](C)CCCCCCCC(=O)O ; CCC[C@H](C)/C=C(/C)C/C(C)=C/[C@H](C)CCCCCCCC(=O)O ; CCC[C@H](C)/C=C(/C)C/C(C)=C/[C@@H](C)CCCCCCCC(=O)O]

Exemplary Embodiment 61. The method of exemplary embodiment 54, wherein binding is assessed by detecting activity of the targeted receptor in a biological assay.

Exemplary Embodiment 62. The method of exemplary embodiment 61, wherein the biological assay is a β-arrestin assay.

Exemplary Embodiment 63. The method of any one of exemplary embodiments 54-62, further comprising creating a model that shows conformation of the target fatty acid bound to the fatty acid receptor, wherein the model is based on the assessment of which of the mimetics of the set bound to the fatty acid receptor.

Exemplary Embodiment 64. The method of exemplary embodiment 63, wherein the model comprises a lattice that depicts the three-dimensional space of the target fatty acid bound to the fatty acid receptor.

Exemplary Embodiment 65. The compound or pharmaceutically acceptable salt of exemplary embodiment 1, wherein the compound has a structure of Formula (I-A):

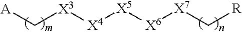

wherein X³ is a bond, an alkyl, or a conformational constraint;
X⁴ is a bond or a flexible subunit;
X⁵ is a conformational constraint;
X⁶ is a bond or a flexible subunit; and
X⁷ is a bond, an alkyl, or a conformational constraint.

Exemplary Embodiment 66. The compound or pharmaceutically acceptable salt of exemplary embodiment 65, wherein at least one of X³ or X⁷ is a conformational constraint.

Exemplary Embodiment 67. The compound or pharmaceutically acceptable salt of exemplary embodiment 65, wherein at least one of X³ or X⁷ is selected from (i) to (x):

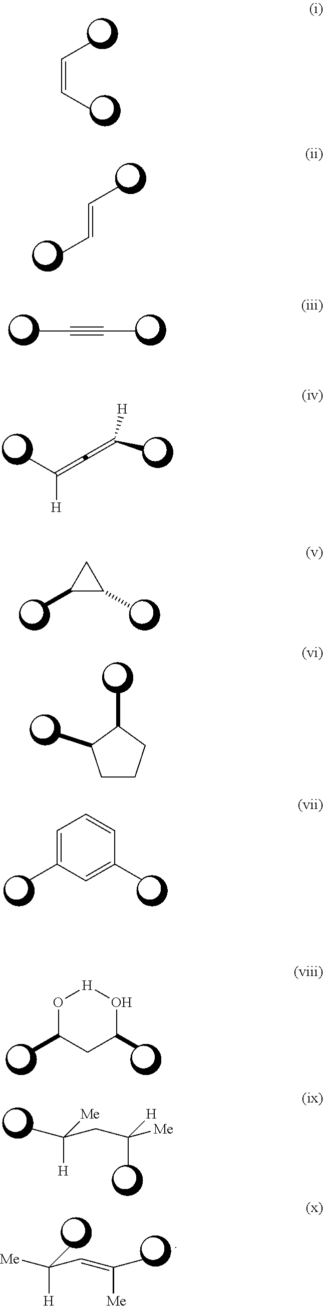

Exemplary Embodiment 68. The compound or pharmaceutically acceptable salt of exemplary embodiment 65, wherein at least one of X³ or X⁷ is a conformational constraint comprising a ring system.

Exemplary Embodiment 69. The compound or pharmaceutically acceptable salt of exemplary embodiment 68, wherein at least one of X³ or X⁷ is a cycloalkyl.

Exemplary Embodiment 70. The compound or pharmaceutically acceptable salt of exemplary embodiment 69, wherein at least one of X³ or X⁷ is selected from a stereodefined disubstituted cyclopentane, cyclopropane, cyclohexane, tetrahydrofuran, tetrahydropyran, g-lactone, g-lactam, regiodefined di- or tri-substituted pyrone, pyridone, pyridine, or benzene.

Exemplary Embodiment 71. The compound or pharmaceutically acceptable salt of any one of exemplary embodiments 65-70, wherein X⁴ and X⁶ are independently C₁₋₆-alkylene, such as a C₁₋₃-alkylene (e.g., methylene, ethylene, or propylene), preferably C₁ (e.g., methylene).

1. A compound of Formula (I): 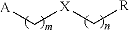
or a pharmaceutically acceptable salt thereof; wherein:
A comprises a polar head group;
X comprises a conformationally constrained core comprising two or more conformational constraints and at least one optional flexible subunit;
R comprises a non-polar group; and
each of m and n is independently an integer from 1 to 25, or from 1 to 19, or from 1 to 11, or from 1 to 7, preferably m is 1, 3, 5, 7, 9, 11, 13, 15, 17, 19, 21, 23 or 25 and preferably n is 1, 3, 5, 7, 9, 11, 13, 15, 17, 19, 21, 23 or 25 and preferably m+n is 6, 7, 8, 9, 10, 11, 12, 13, 14, 15, 16, 17, 18, 19, 20, 21, 22, 23, 24 25, 26, 27, 28, 29, 30, 31, 32, 33, 34, 35, 36, 37, 38, 39, or 40.

2. The compound or pharmaceutically acceptable salt of claim 1, wherein the flexible subunit is a methylene subunit.

3. The compound or pharmaceutically acceptable salt of claim 1, wherein X comprises (ix)

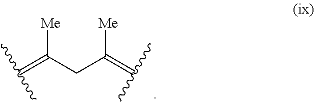 [SMILES: *=C(C)CC(=*)C]

4. The compound or pharmaceutically acceptable salt of claim 1, wherein each the two or more conformational constraints is independently selected from the group consisting of (x), (x′), (x″), and (x′″):

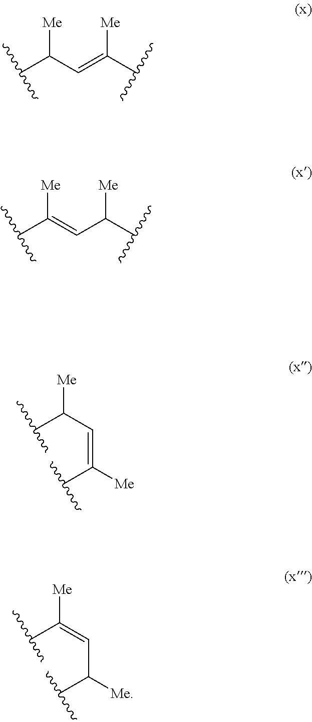 [SMILES: */C(C)=C/C(*)C ; */C(C)=C/C(*)C ; */C(C)=C\C(*)C ; */C(C)=C\C(*)C]

5. The compound or pharmaceutically acceptable salt of claim 4, wherein the two or more conformational constraints are joined by a C₁₋₆-alkylene, such as a C₁₋₃-alkylene (e.g., methylene, ethylene, or propylene), preferably C₁ (e.g., methylene).

6. The compound or pharmaceutically acceptable salt of claim 1, wherein A comprises A²⁻R¹⁵⁻, wherein A² is a polar head group, and R¹⁵ is a bond, a C₁₋₆-alkyl, or a C₂-C₆ alkenyl.

7. The compound or pharmaceutically acceptable salt of claim 6, wherein A² is a carboxylic acid, a carboxyl ester, an amide, a ketone, a phosphonic acid, a phosphinic acid, a sulfonic acid, a sulfinic acid, a sulfonamide, an acyl-sulfonamide, a hydroxamic acid, a hydroxamic ester, a sulfonylurea, an acylurea, a tetrazole, a thiazolidinedione, an oxazolidinedione, an oxadiazolone, a thiadiazolone, an oxathiadiazole oxide, an oxadiazolethione, an isoxazole, a tetramic acid, a cyclopentane diones, a phenol derivative, a squaric acid derivative, or a salt thereof.

8. The compound or pharmaceutically acceptable salt of claim 1, wherein the compound has a structure of Formula (I-A): 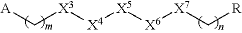
wherein X³ is a bond, an alkyl, or a conformational constraint;
X⁴ is a bond or a flexible subunit;
X⁵ is a conformational constraint;
X⁶ is a bond or a flexible subunit; and
X⁷ is a bond, an alkyl, or a conformational constraint.

9. The compound or pharmaceutically acceptable salt of claim 8, wherein at least one of X³ or X⁷ is a conformational constraint.

10. The compound or pharmaceutically acceptable salt of claim 8, wherein at least one of X³ or X⁷ is selected from (i) to (x):

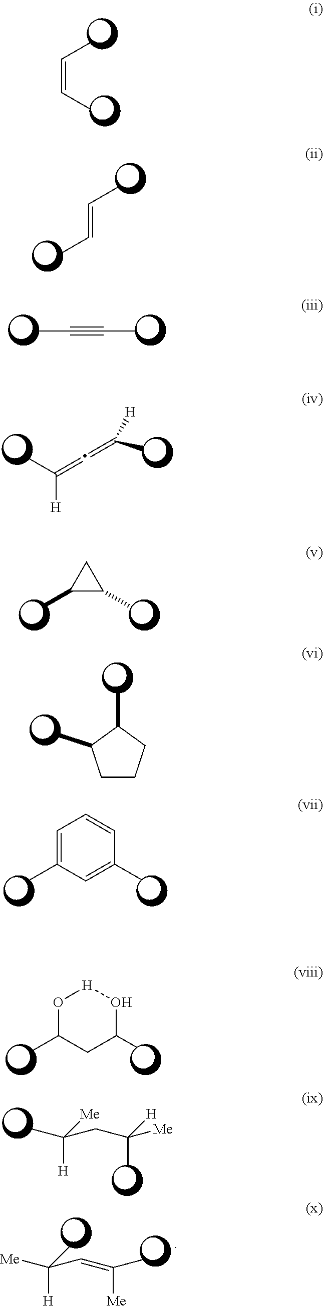

11. The compound or pharmaceutically acceptable salt of claim 8, wherein at least one of X³ or X⁷ is a conformational constraint comprising a ring system.

12. The compound or pharmaceutically acceptable salt of claim 11, wherein at least one of X³ or X⁷ is a cycloalkyl.

13. The compound or pharmaceutically acceptable salt of claim 12, wherein at least one of X³ or X⁷ is selected from a stereodefined disubstituted cyclopentane, cyclopropane, cyclohexane, tetrahydrofuran, tetrahydropyran, g-lactone, g-lactam, regiodefined di- or tri-substituted pyrone, pyridone, pyridine, or benzene.

14. The compound or pharmaceutically acceptable salt of claim 8, wherein X⁴ and X⁶ are independently C₁₋₆-alkylene, such as a C₁₋₃-alkylene (e.g., methylene, ethylene, or propylene), preferably C₁ (e.g., methylene).

15. A compound of Formula (II): 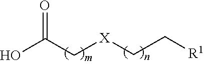
or a pharmaceutically acceptable salt thereof; wherein:
X comprises a constrained core comprising one or more acyclic conformational constraints and at least one optional flexible subunit;
R¹ is a C₁₋₆-alkyl; and
each of m and n is independently an integer from 1 to 25, or from 1 to 19, or from 1 to 11, or from 1 to 7, preferably m is 1, 3, 5, 7, 9, 11, 13, 15, 17, 19, 21, 23 or 25 and preferably n is 1, 3, 5, 7, 9, 11, 13, 15, 17, 19, 21, 23 or 25 and preferably m+n is 6, 7, 8, 9, 10, 11, 12, 13, 14, 15, 16, 17, 18, 19, 20, 21, 22, 23, 24 25, 26, 27, 28, 29, 30, 31, 32, 33, 34, 35, 36, 37, 38, 39, or 40;
provided that the compound is not a natural product and/or
provided that at least one conformational constraint is a conformational constraint other than

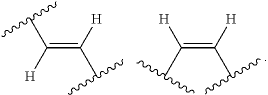

16. The compound or pharmaceutically acceptable salt of claim 15, wherein the constrained core comprises two or more acyclic conformational constraints.

17. The compound or pharmaceutically acceptable salt of claim 15, wherein each of the one or more acyclic conformational constraints is (x), (x′), (x″), or (x′″): 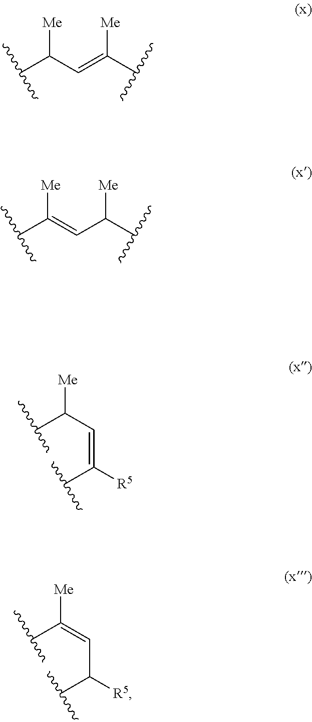
wherein R⁵ is hydrogen or C₁₋₆-alkyl.

18. (canceled)

19. The compound or pharmaceutically acceptable salt of claim 15, wherein X comprises:

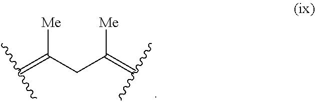 [SMILES: *=C(C)CC(=*)C]

20. The compound or pharmaceutically acceptable salt of claim 15, wherein X is: 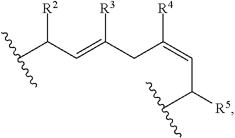
wherein:
R² is C₁₋₆-alkyl;
R³ is C₁₋₆-alkyl;
R⁴ is C₁₋₆-alkyl; and
R⁵ is hydrogen or C₁₋₆-alkyl.

21. A compound of Formula (III): 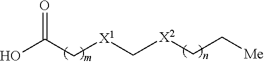
or a pharmaceutically acceptable salt thereof; wherein:
X¹ is 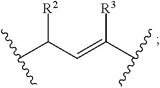
R² is C₁₋₆-alkyl;
R³ is C₁₋₆-alkyl;
X² is 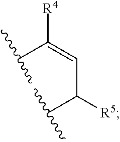
R⁴ is C₁₋₆-alkyl;
R⁵ is hydrogen, deuterium, or C₁₋₆-alkyl; and
each of m and n is independently an integer from 1 to 25, or from 1 to 19, or from 1 to 11, or from 1 to 7, preferably m is 1, 3, 5, 7, 9, 11, 13, 15, 17, 19, 21, 23 or 25 and preferably n is 1, 3, 5, 7, 9, 11, 13, 15, 17, 19, 21, 23 or 25 and preferably m+n is 6, 7, 8, 9, 10, 11, 12, 13, 14, 15, 16, 17, 18, 19, 20, 21, 22, 23, 24 25, 26, 27, 28, 29, 30, 31, 32, 33, 34, 35, 36, 37, 38, 39, or 40.

22. The compound or pharmaceutically acceptable salt of claim 21, wherein X¹ is (x.1) or (x.2): 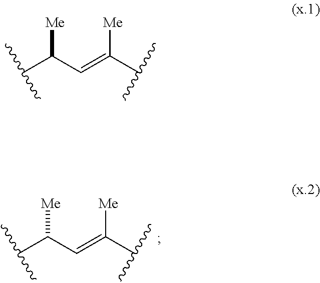 [SMILES: */C(C)=C/[C@@H](*)C ; */C(C)=C/[C@H](*)C]
and
X² is (x′″.1) or (x′″.2):

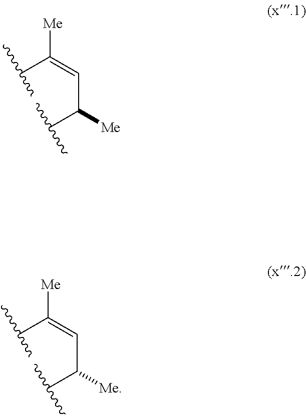 [SMILES: */C(C)=C\[C@H](*)C ; */C(C)=C\[C@@H](*)C]

23. The compound or pharmaceutically acceptable salt of claim 20, wherein each of R², R³, and R⁴ are methyl or a partially or fully deuterated methyl, and R⁵ is hydrogen, deuterium, methyl, or a partially or fully deuterated methyl.

24. The compound or pharmaceutically acceptable salt of claim 20, wherein each of R², R³, and R⁴ are methyl or a partially or fully deuterated methyl, and R⁵ is hydrogen or deuterium.

25. The compound or pharmaceutically acceptable salt of claim 20, wherein each of R², R³, and R⁴ are methyl or a partially or fully deuterated methyl, and R⁵ is C₁₋₆-alkyl.

26. The compound or pharmaceutically acceptable salt of claim 20, wherein each of R², R³, R⁴, and R⁵ are methyl or a partially or fully deuterated methyl.

27. (canceled)

28. (canceled)

29. (canceled)

30. A compound or a pharmaceutically acceptable salt thereof, wherein the compound is selected from one of Compounds 1-A to 4-F:

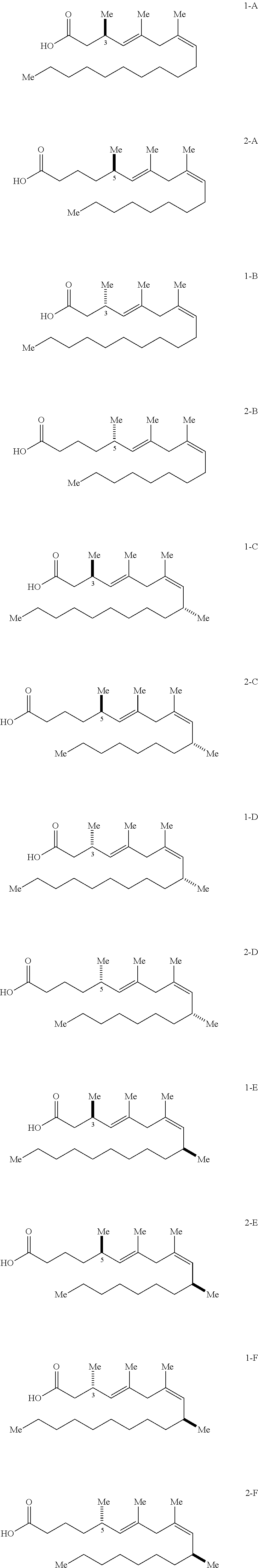

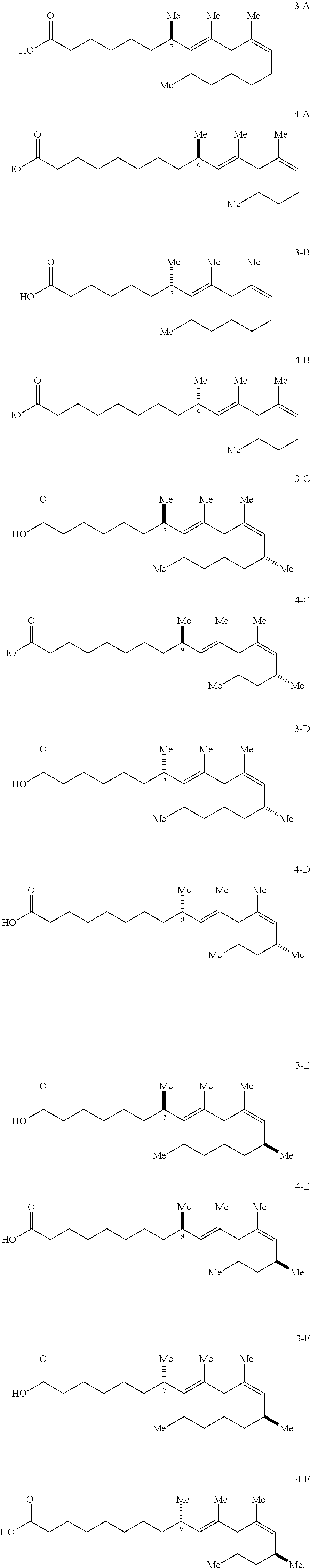

31. A method for modulating activity of GPR40 and/or GPR120, the method comprising contacting GPR40 and/or GPR120 with the compound or the pharmaceutically acceptable salt of claim 1.

32. A method for treating a metabolic disease (e.g., type 2 diabetes) in a subject in need thereof, the method comprising administering to the subject the compound or the pharmaceutically acceptable salt of claim 1.

33. A method for treating obesity in a subject in need thereof, the method comprising administering to the subject the compound or the pharmaceutically acceptable salt of claim 1.

34. A kit or set comprising two or more fatty acid mimetics, wherein each of the fatty acid mimetics is:
a compound of claim 1 or a pharmaceutically acceptable salt thereof.

35. (canceled)

36. (canceled)

37. (canceled)

38. (canceled)

39. (canceled)

40. A method for making a compound of claim 1, the method comprising:
coupling an alkyne of Formula (IV): 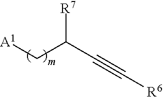
wherein A¹ comprises a polar head group, or a head group protected with a protecting group;
R⁶ is a C₁₋₆-alkyl;
R⁷ is hydrogen or a C₁₋₆-alkyl;
with an allylic alcohol of Formula (V): 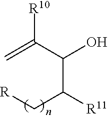
wherein R comprises a non-polar group;
R¹⁰ is hydrogen or a C₁₋₆-alkyl;
R¹¹ is hydrogen or a C₁₋₆-alkyl; and
each of m and n is independently an integer from 1 to 25, or from 1 to 19, or from 1 to 11, or from 1 to 7.

41. (canceled)

42. (canceled)

43. (canceled)

44. (canceled)

45. (canceled)

46. (canceled)

47. A method of conformational profiling comprising:
preparing a set of mimetics of the target fatty acid, wherein the set of mimetics comprises at least one compound of claim 1 or a Pharmaceutically acceptable salt thereof; and
assessing the functional activity of each member of the set of fatty acid mimetics at a desired fatty acid receptor (e.g., GPR40).

48. (canceled)

49. (canceled)

50. (canceled)

51. (canceled)
